(19) United States
(12) Patent Application Publication
(10) Pub. No.: US 2023/0374492 A1
(43) Pub. Date: Nov. 23, 2023

(54) PROCESS FOR SELECTION OF APTAMERS, RIBOSWITCHES AND DESOXYRIBOSWITCHES

(71) Applicant: Centre National de la Recherche Scientifique (CNRS), Paris (FR)

(72) Inventors: Marc BOUDVILLAIN, Combreux (FR); Emilie SOARES, Joue-Les-Tours (FR); Annie SCHWARTZ, Saint Cyr En Val (FR); Mildred DELALEAU, Estouy (FR)

(73) Assignee: Centre National de la Recherche Scientifique (CNRS), Paris (FR)

(21) Appl. No.: 18/029,964

(22) PCT Filed: Oct. 5, 2021
(86) PCT No.: PCT/EP2021/077367

§ 371 Date: Apr. 3, 2023

(30) Foreign Application Priority Data
Oct. 7, 2020 (EP) 20306167.6

(51) Int. Cl. C12N 15/10; C12N 15/115

(52) U.S. Cl. CPC C12N 15/1048; C12N 15/115; C12N 2310/16; C12N 2320/13

(57) ABSTRACT

The invention relates to a process for selecting aptamers substrates of one helicase enzyme, comprising the implementation of a helicase SELEX process comprising several cycles, wherein each cycle comprises the following steps: a) Providing a library of nucleic acid duplex constructs comprising one nucleic acid strand containing a random sequence of 10 to 100 nucleotides framed by fixed sequences at each end; b) Incubation of said library with said helicase in appropriate conditions for the dissociation of certain duplex constructs by the helicase, resulting in release of the aptamers substrates of the helicase; c) Isolation and amplification of said aptamers substrates of the helicase; d) Creation of a novel library of nucleic acid duplex constructs enriched in duplex constructs comprising aptamers substrates of the helicase.

### FIELD OF THE INVENTION

The present invention is related to a process of selection of aptamers, riboswitches and desoxyriboswitches, based on the functional activity of said aptamers and switches instead of their structural affinity.

The present invention also relates to aptamers and switches (riboswitches and desoxyriboswitches) obtained with this process, and their use as reporters of the activity of a helicase, or as bio-sensors of presence of a specific compound.

The invention also relates to reporter assays using the same.

### BACKGROUND OF THE INVENTION

Nucleic acid molecules can adopt complex three-dimensional folds, and therefore are endowed with molecular recognition capabilities equivalent to those of proteins. Single-stranded RNA or DNA whose 3D conformation generates a specific interaction pocket/surface for a ligand are designated as “aptamers”.

Aptamers present several advantages and raise great interest among the scientific community. In particular, they could be used for the same applications than antibodies.

Aptamers are also part of “riboswitches”, RNA compounds having the ability to modulate gene expression. Riboswitches are gene expression modulators, that are made up of (i) an aptamer able to bind a specific inducer, and (ii) another RNA motif, designated as the “expression platform” that is allosterically (structurally) connected to the aptamer. Inducers may be metabolites, vitamins, amino acids or ions. Sensing of the absence or presence of the inducer by the aptamer is allosterically translated into formation of one of two mutually exclusive (inactive/active) conformations of the expression platform, which in turn leads to a specific regulation of a downstream target gene in function of the presence/absence of said inducer.

Natural riboswitch specimens have been discovered in various organisms from bacteria, archaea, and eukaryotes. Distinct classes of riboswitches have been identified and are shown to selectively recognize activating compounds. For example, coenzyme B12, glycine, thiamine pyrophosphate (TPP), and flavin mononucleotide (FMN) activate riboswitches present upstream from genes encoding key enzymes in metabolic or transport pathways of these compounds. Another class of riboswitches is activated with guanine, a purine-derived nucleobase.

Interestingly, riboswitches may be used as bio-sensors detecting absence or presence of activating compounds also called “inducers”.

Just as natural riboswitches can regulate gene expression in response to specific ligands, synthetic riboswitches can be engineered to repress or activate gene expression in a ligand-dependent fashion. This faculty makes riboswitches fabulous tools that can be used in industry, medicine, pharmacy and other fields.

Methods for generating synthetic aptamers and riboswitches have been disclosed. In particular, synthetic aptamers may be selected in vitro from random libraries, by using the SELEX method described below.

The SELEX methodology (for Systematic Evolution of Ligands by EXponential enrichment) consists in the combination of selection of aptamers from a pool of single-stranded RNAs or DNAs, which interact with a target in a desirable manner, and the amplification of those selected aptamers. The pool comprises single-stranded RNAs or DNAs where the central part has a randomized nucleic acid sequence, and the external parts have a fixed sequence (used for PCR or RT-PCR amplification of DNA or RNA, respectively). In a selection step, the single-stranded RNAs or DNAs with the highest affinity for the target are partitioned from those with lesser affinity for the target. Those single-stranded nucleic acids selected as having the relatively higher affinity for the target are then amplified to create a new candidate mixture that is enriched in nucleic acids having a relatively higher affinity for the target. Iterative cycling of the selection and amplification steps allows an enrichment of the most promising single-stranded nucleic acids. A final step of sequencing of the enriched sequences allows the identification of novel synthetic aptamers specific of the used target. This SELEX process is described for example in U.S. Pat. Nos. 5,475,096 and 5,270,163. Improvements of the SELEX process have been disclosed in U.S. Pat. Nos. 5,707,796, 5,763,177, 6,001,577, 5,580,737, 5,567,588 and 6,706,482.

As an example, this method has been successfully used for selecting RNA aptamers against SARS coronavirus helicase (nsP10), from a RNA library containing random sequences of 40 nucleotides, after 15 successive rounds of SELEX process; selected aptamers are good candidates for use as anti-SARS coronavirus agents (22).

Nevertheless, this SELEX method is limited by various disadvantages.

In most approaches, the target has to be immobilized on a surface, which therefore limits the choice of the possible targets.

Secondly, aptamers are selected indiscriminately for binding to any available surface/pocket on the target. For big targets such as helicases, aptamer binding to a surface/pocket far from the enzyme active site may not translate into modulation of activity.

Thirdly, this technology tends to select rigid conformational structures of RNA or DNA strands, with a high structural affinity for the target, but not suitable for incorporation into a biosensor that requires structural flexibility.

### SUMMARY OF THE INVENTION

The present invention concerns a process for selecting nucleic acid motifs, in particular aptamers substrates of specific helicase enzymes, and switches able to modulate specifically the activity of an helicase enzyme.

In the present specification, the term “switch” refers to any DNA or RNA structure able to control the activity of a helicase in an inducer-dependent manner, in vitro or in vivo. This switch may either consist of RNA and is therefore designated as “riboswitch”, or consist of DNA and is therefore designated as “desoxyriboswitch”.

This process is based on a molecular evolution process of SELEX type, improved with a step of functional selection of nucleic acid motifs.

Instead of the usual affinity selection step, a functional selection step is performed, based on the enzymatic activity of an helicase enzyme. This functional selection step allows the enrichment of nucleic acid motifs that promote the helicase activity.

In the process for selecting switches, nucleic acid motifs are selected on their ability to promote the helicase activity only in the presence of an activating compound, hereafter designed as “inducer”.

This process of selection of nucleic acid aptamers and switches is a modified SELEX process that is designated hereafter as the “Helicase SELEX” or “Helicase-SELEX” process.

In a first aspect, the present invention concerns a process for selecting aptamers substrates of one helicase enzyme, comprising the implementation of a helicase SELEX process comprising several cycles, wherein each cycle comprises the following steps:
a) Providing a library of nucleic acid duplex constructs comprising one nucleic acid strand containing a random sequence of 10 to 100 nucleotides framed by fixed sequences at each end;
b) Incubation of said library with said helicase in appropriate conditions for the dissociation of certain duplex constructs by the helicase, resulting in release of the aptamers substrates of the helicase;
c) Isolation and amplification of said aptamers substrates of the helicase;
d) Creation of a novel library of nucleic acid duplex constructs enriched in duplex constructs comprising aptamers substrates of the helicase.

In a second aspect, the present invention concerns a process for selecting switches stimulating the activity of one helicase enzyme in response to the presence of a specific inducer, comprising the implementation of a helicase SELEX process comprising several cycles, wherein each cycle comprises the following steps:
a) Providing a library of nucleic acid duplex constructs comprising one nucleic acid strand containing a random sequence of 10 to 100 nucleotides framed by fixed sequences at each end;
b1) Incubation of said library with said helicase and said specific inducer in appropriate conditions for the dissociation of certain duplex constructs by the helicase, resulting in release of the switches comprising the sequences that are substrates of the helicase in presence of said inducer; and/or
b2) Incubation of said library with said helicase without any inducer for retention of switch-containing duplex constructs not dissociated by said helicase in absence of said inducer, and elimination of duplex constructs dissociated by said helicase in absence of said inducer;
c) Isolation and amplification of said switches;
d) Creation of a novel library of nucleic acid duplex constructs enriched in duplex constructs comprising switches modulating the activity of the helicase in presence of a specific inducer,
and wherein at least one cycle comprises at least one step (b1).

The present invention also relates to isolated aptamers substrates of a helicase obtained by the process as described above.

The present invention also relates to isolated switches (riboswitches or desoxyriboswitches) modulating the activity of a helicase in response to the presence of a specific inducer, obtained by the process as described above.

Another object of the present invention is a genetic construction comprising a switch selected with the process previously described, and an expression cassette. This genetic construction may be in particular a reporter system comprising a reporter gene in the expression cassette.

Another object of the present invention is a method for detecting a compound of interest, using the reporter system as described above, wherein the switch is responsive to the presence of said compound of interest.

### BRIEF DESCRIPTION OF THE FIGURES

FIG. 1. Diagram illustrating the steps of the helicase SELEX process for selecting RNA aptamers

In the first step of the Helicase-SELEX cycle, a DNA template containing a random region (50 base pairs in the pictured example) framed by fixed sequences FWD and REV is transcribed to yield a library of single-stranded RNA (ssRNA) strands. Pairing of a biotinylated oligonucleotide to the REV region of the ssRNAs yields a library of duplexes, which are then immobilized on streptavidin beads. The beads are incubated with the helicase of interest (here Rho) in an appropriate buffer containing NTP (here ATP) for a given time. The ssRNA strands released in the supernatant are selectively recovered and amplified by RT-PCR to yield a new DNA template library enriched in aptamer sequences.

The new library can be used in a new Helicase-SELEX cycle and the process repeated iteratively, with increasing stringency (through reduction of incubation time with the helicase for instance), until the library is sufficiently enriched in aptamers (enrichment evaluated through the ability of the corresponding library of duplexes to elicit a strong helicase activity; see FIG. 2).

FIG. 2. Measurement of Rho helicase activity as a function of time, for libraries of duplexes: original (R0) or obtained after 1 (R1), 3 (R3), 5 (R5) or 7 cycles/rounds (R7) of Helicase SELEX.

The “unwound fraction” corresponds to the quantity of dissociated duplexes, i.e. the quantity of single-stranded RNA released in the supernatant under the helicase action.

FIG. 3. Diagram illustrating the steps of the helicase SELEX process for selecting DNA aptamers

Most steps are common with the Helicase-SELEX process implemented for selecting RNA aptamers. The main difference is the step of amplification that is carried out by asymmetric PCR and directly generates single stranded DNAs. In the diagram, Upf1 is used as an example of helicase able to unwind DNA.

FIG. 4. Process of selection of switches using the helicase SELEX process

(A) Diagram illustrating the steps of the helicase SELEX process for selecting switches sensitive to an inducer

Inducible switches (in this figure, riboswitches) are obtained by combining selection (in presence of inducer) and counter-selection (in absence of inducer) steps in the Helicase-SELEX procedure. Each Helicase-SELEX cycle may contain a single selection or counter-selection step; selection and counterselection cycles are mixed during the iterative enrichment procedure. Alternatively, a counter-selection step and a selection step may be combined and performed sequentially in any single Helicase-SELEX cycle.

(B) Method of selection of inducer-activated switches

Sequences able to form a catalytically efficient interaction with the helicase enzyme in presence of the inducer, promote duplex unwinding by the helicase and can be selectively recovered from the supernatant (selection step). To separate inducer-dependent sequences from constitutively active sequences, a counter-selection step in absence of inducer is necessary. Constitutively active sequences are released in the supernatant while inducer-dependent sequences remain bound to the beads (the beads fraction is thus collected in this case). The inducer-activated sequences are inactive in absence of inducer, because either they cannot bind the helicase (as depicted) or they interact with the helicase enzyme in a catalytically inefficient manner (not illustrated).

FIG. 5. Helicase-SELEX for selection of riboswitches

(A) A library of RNA-DNA duplexes is constructed with a random sequence of 80 nucleotides framed by fixed sequences at each end. Sequences of FWD, REV and SEL primers, and fixed sequences used in the library, are listed in Table 1.

(B) Measurement of Rho helicase activity as a function of time, for libraries of duplexes obtained after 3 (R3), 6 (R6), 7 (R7), 8 (R8) and 9 (R9) cycles. On top, a schema illustrates the dissociation of duplexes under the activity of Rho helicase, in presence of ATP and an inducer (5-HT).

FIG. 6. Response to serotonin, the inducer compound, according to Helicase-SELEX cycles

(A) Top: Fraction of dissociated RNA-DNA duplexes after 30 min of reaction in the presence of Rho and ±10 mM serotonin (5-HT);

Bottom: Reactivity gain in the presence of serotonin (1 or 10 mM as shown on the right) measured after 30 min of helicase reaction.

(B) Overall helicase activity of the RNA-DNA library most sensitive (R13) to the presence of 10 mM Serotonin.

FIG. 7. Main features of the control pFACS-aRut-mgtA-Tac1 plasmid

The sequence of the pTac-sfGFP leader region containing a strong Rho-dependent transcription termination signal is shown above the plasmid map. It is identified as SEQ ID NO. 20. The aRut sequence (boxed) is deleted in control plasmid pFACS-RutLess-mgtA-Tac1. In the R13 dual reporter plasmid library, the aRut sequence is replaced by the N80-derived sequences evolved by Helicase-SELEX after 13 rounds. The site of long-lived transcriptional pausing in the mgtA leader region (1) is identified by a black star. The mgtA leader region is absent from the plasmid derivatives of the tsp-less series. DsRed-Express2 is a reporter gene used for normalisation. GFPsf is the reporter gene for assaying the activation of the system.

FIG. 8. Helicase activity of isolated sequences

Sequences from the R13 library were randomly selected. The sequences from R21 library are the 4 most abundant sequences detected by NGS sequencing=>sequences R13-C37, R21-49050 and R21-30360 seem the most interesting.

FIG. 9. Schematic illustration of an automated Helicase-SELEX procedure Reagents are stored in dedicated vessels on temperature-controlled stations of the robotic platform (dotted box on bottom left). The Helicase-SELEX reaction mixtures are sequentially assembled with a robotic pipetting arm in a 96-deep well SBS plate installed on a temperature-controlled shaker (second row from top). This format allows the processing of up to 8 samples in parallel (diagram depicts the case for 4 samples). Each sample is handled in a distinct well of the same plate column; sample reactions are performed in distinct wells of the same plate row, from left (transcription) to right (unwinding reaction); reaction volumes are dispatched in several wells if necessary. Purifications by magnetic separation (for activation and washings of beads, purification of bead-immobilized duplexes after transcription, recovery or supernatant or beads after, respectively, a selection or counter-selection step in presence of the helicase, etc.) are performed by moving the 96-well SBS plate onto a dedicated magnet (third row from top). If a counter-selection step is performed (optional counter cycle on the diagram), bead-immobilized duplexes are recovered after a first unwinding reaction performed under counter-selection conditions, then washed, and used directly in a second unwinding reaction performed under “selection” conditions. For fast selection reaction steps, samples are stored transiently at room temperature in a dedicated 96-well plate and processed sequentially (rather than simultaneously) in the “reaction” plate installed on the temperature-controlled shaker. In this way, the multi-channel pipetting arm can be preloaded with both helicase initiation and quench mixes (in distinct channels), thereby eliminating time lags inherent to tips change and reagents loading. Supernatants recovered after selection step reactions are mixed with reagents for RNA solid phase extraction (SPE) in dedicated vessels (tubes) before being loaded on SPE columns installed on a vacuum filtration station. SPE eluates are directly collected in a PCR microplate wherein both the reverse transcription (RT) and PCR reaction mixtures are assembled. The RT and PCR reactions are performed in a robot-controlled thermocycler. Transfers of the 96-deep well SBS and PCR plates between the various stations of the robot worktable (thermoshaker, magnet, SPE module, thermocycler, etc.) are performed with a dedicated robotic arm.

FIG. 10: Helicase-SELEX with a library of natural sequences

(A) Schematic description of the strategy used to prepare RNA:DNA duplexes containing natural ssRNA sequences. Genomic DNA from E. coli was PCR amplified with a pair of partially randomized primers as described (2). A second PCR round was used to equip the resulting gDNA fragments with the full T7 promoter and REV region sequences and fragments in the appropriate size range (containing a 50 to 100 bp genomic sequence) were purified by native PAGE. The purified fragments were then transcribed with T7 RNA polymerase and the resulting transcripts hybridized with DNA strands to form RNA-DNA duplexes containing natural ssRNA sequences.

(B) Measurement of Rho helicase activity as a function of time, for libraries of duplexes containing natural E. coli sequences: original (R0) or obtained after 3 cycles/rounds (R3)

FIG. 11. Use of a biosensor based on a desoxyriboswitch selected with Helicase SELEX process, combined with a fluorophore-quencher couple

Switch-containing duplexes evolved by helicase SELEX can be transformed into simple fluorescent biosensors. This can be achieved, for instance, by linking a fluorescent dye to one of the duplex strand and a molecular quencher to the complementary strand, in structural proximity to the fluorophore in the context of the duplex double helix, as depicted. In absence of the cognate inducer (the analyte of interest, depicted by a star) in the analyzed sample, the helicase cannot unwind the duplex; the quencher thus remains in close proximity to the fluorophore and efficiently quenches its fluorescence (quenched fluorescence state). In the presence of the analyte, the helicase disrupts the duplex, thereby physically separating the fluorophore-quencher pair and triggering an increase in fluorescence (enhanced fluorescence state). Biosensors based on DNA duplexes evolved by Helicase SELEX (FIG. 3) could prove particularly useful to probe analytes in complex or harsh media/matrices, as they would be much less sensitive to degradation than RNA-containing duplexes would be.

FIG. 12. Reporter activity governed by serotonin-dependent riboswitches in E. coli cells

The GFP:dsREDexpress2 reporter fluorescence ratio of E. coli cells bearing tsp-less dual reporter plasmids were determined by flow cytometry in presence (+5-HT) or absence (−5-HT) of 10 mM serotonin. The histograms show the distributions of cells as a function of the reporter fluorescence ratio. Cells carrying control plasmids without sequence insert (top) or with the aRut or iRut sequence in front of the GFP gene (middle row) display similar GFP:dsREDexpress2 ratio distributions in presence and absence of serotonin (≈ symbol). By contrast, cells carrying plasmids with a riboswitch sequence in front of the GFP gene (bottom row) display GFP:dsREDexpress2 ratios that decrease significantly in presence of serotonin (black arrows).

### DETAILED DESCRIPTION OF EMBODIMENTS OF THE INVENTION

Unless stated otherwise, the following terms and phrases as used herein are intended to have the following meanings:

As used herein, “nucleic acid” means either DNA, RNA, single-stranded or double-stranded, and include nucleic acid molecules with any chemical modifications thereof.

As used herein, the term “aptamer” designates a single stranded RNA or DNA molecule whose 3D conformation is specific for the binding of a ligand.

As used herein, the term “switch” designates a gene expression modulator or an helicase activity modulator consisting of an aptamer able to bind a specific ligand, and of a nucleic acid motif called the “expression platform”. The term “riboswitch” designates a switch made of RNA, the term “desoxyriboswitch” designates a switch made of DNA.

As used herein, the term “helicase” or “helicase enzyme” designates an NTP (Nucleoside triphosphate)-dependent enzyme whose function is to disrupt nucleic acids structures, by separating both annealed nucleic acid strands that constitute the DNA double helix (or helices within RNA-RNA and RNA-DNA complexes) or self-annealed single-stranded DNA or

RNA. Helicases act on the hydrogen bonds existing between nucleotides of each strand, and denature duplexes. Metabolic processes such as translation, transcription, RNA splicing, RNA editing, RNA degradation, and homologous DNA recombination necessitate the action of an helicase.

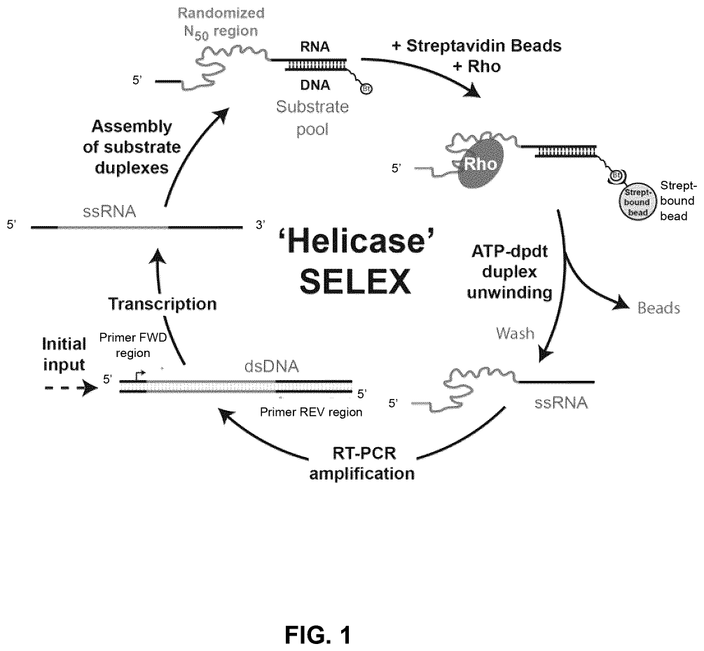

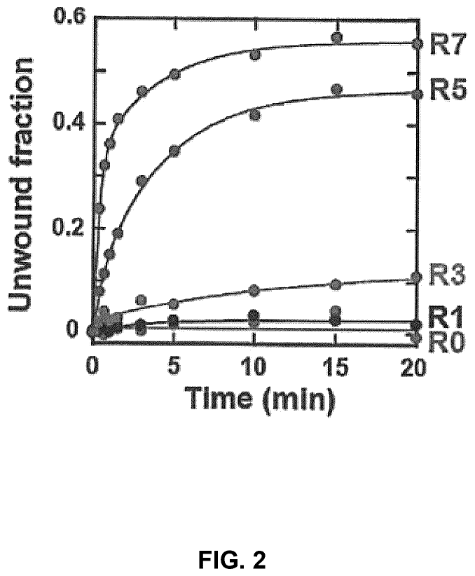

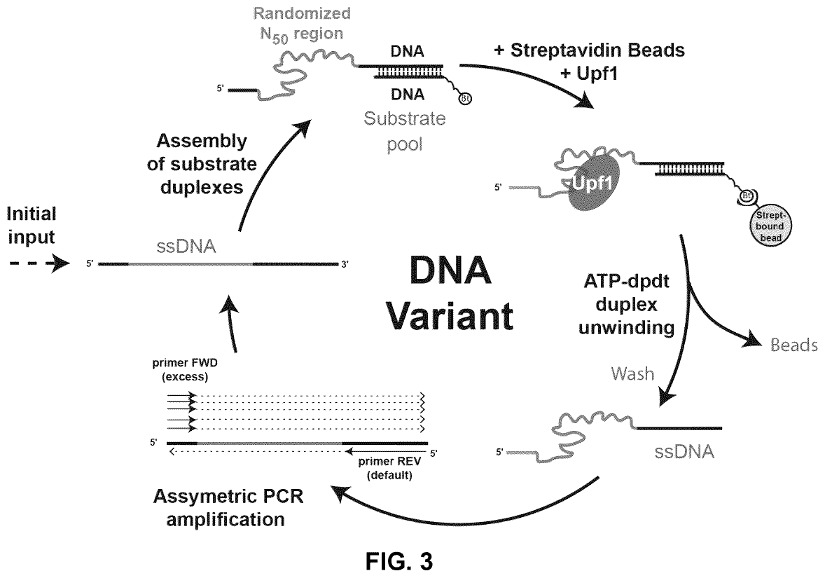

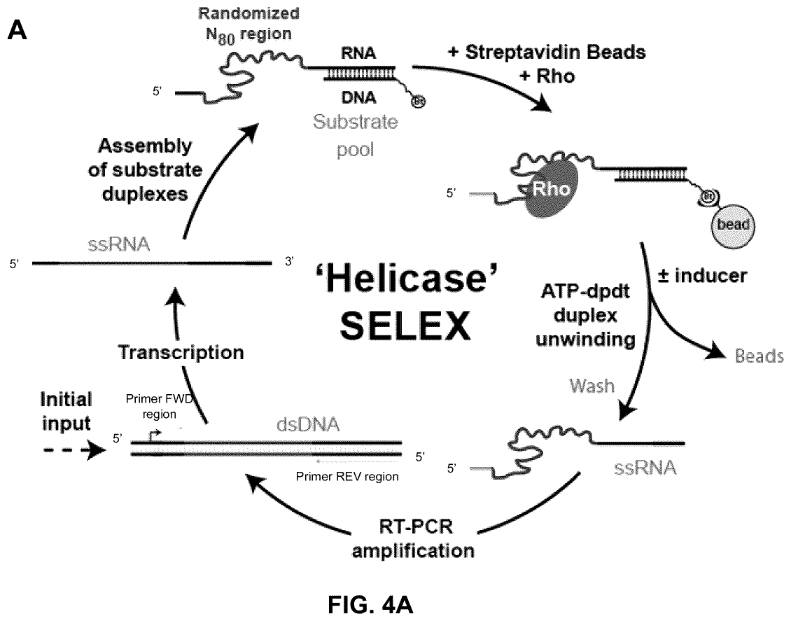

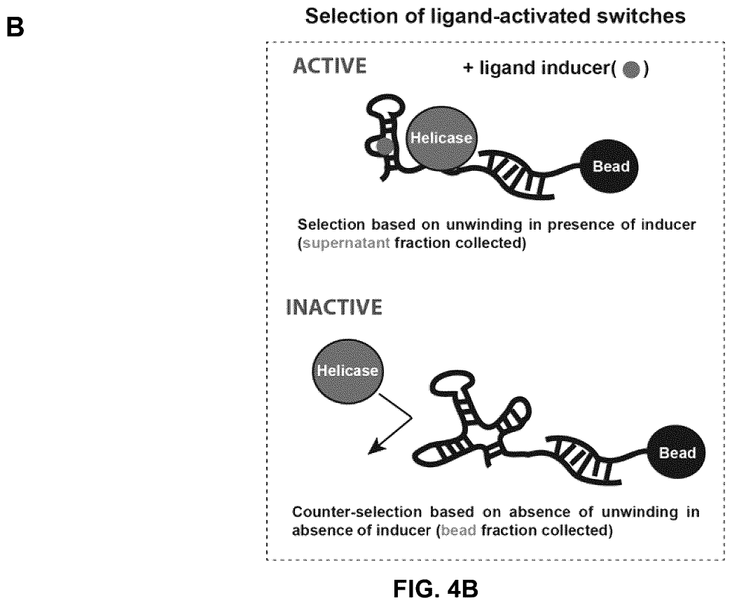

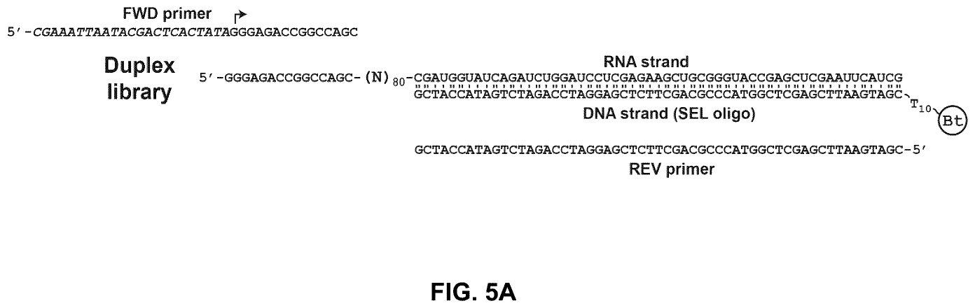

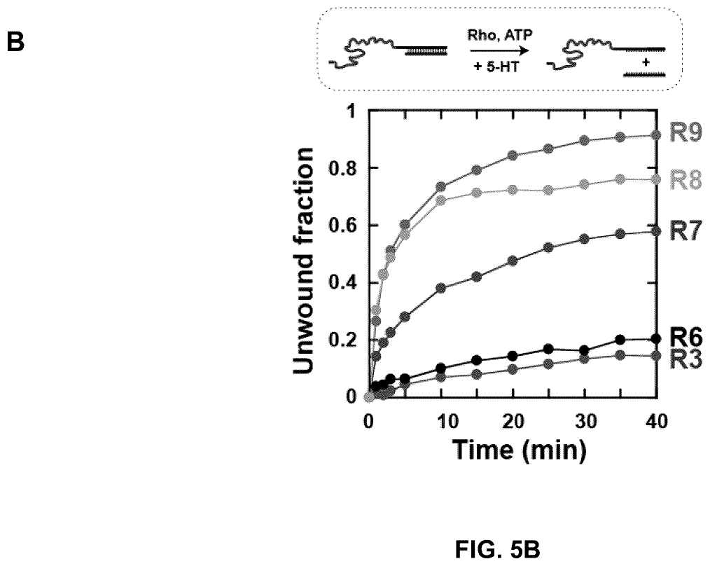

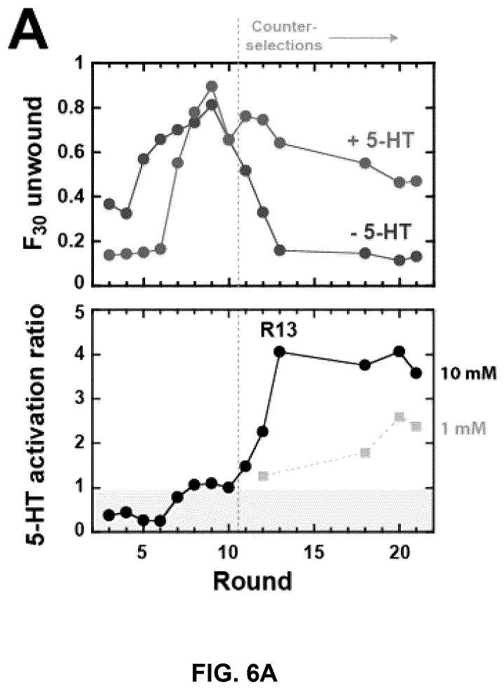

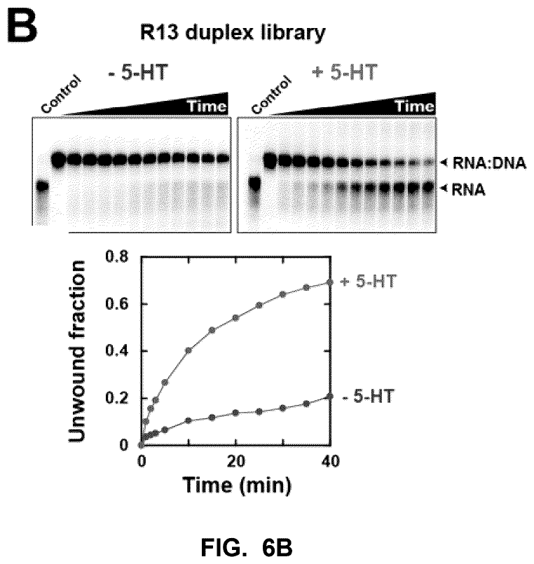

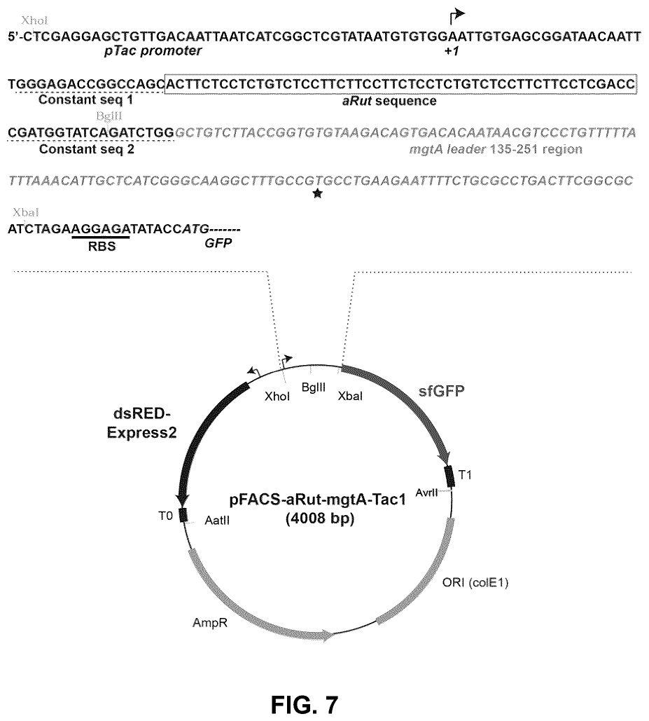

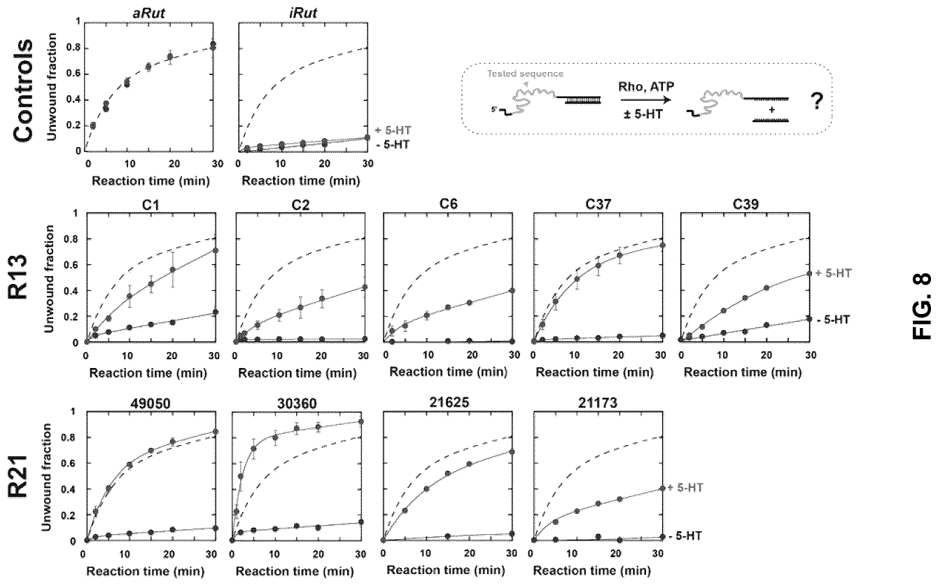

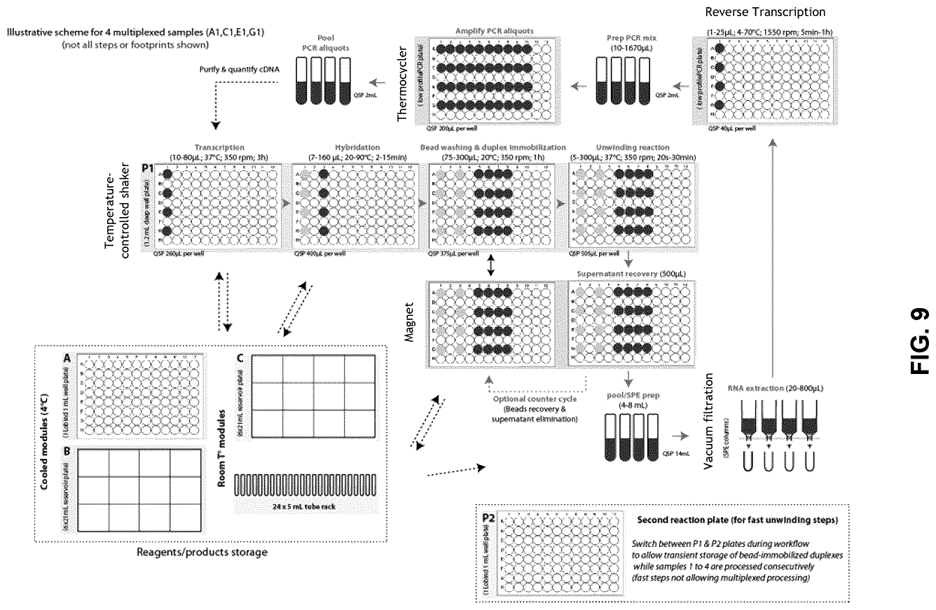

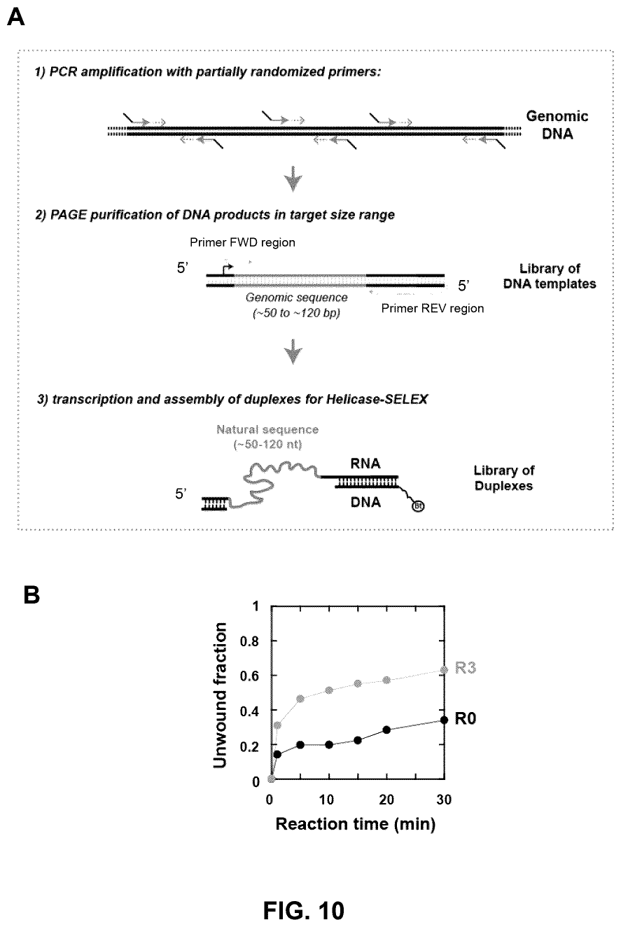

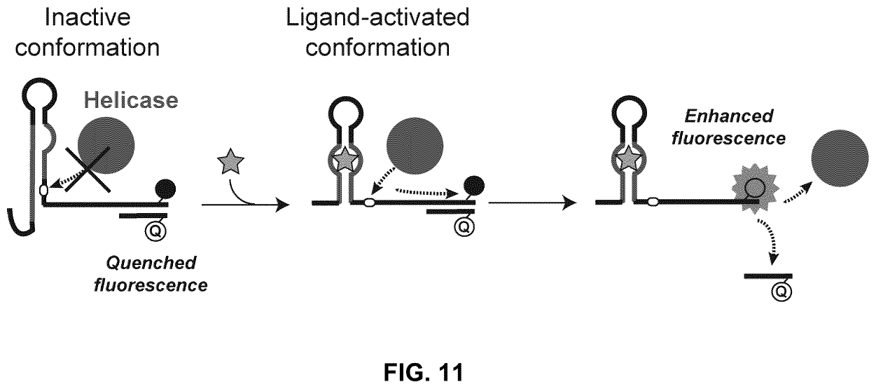

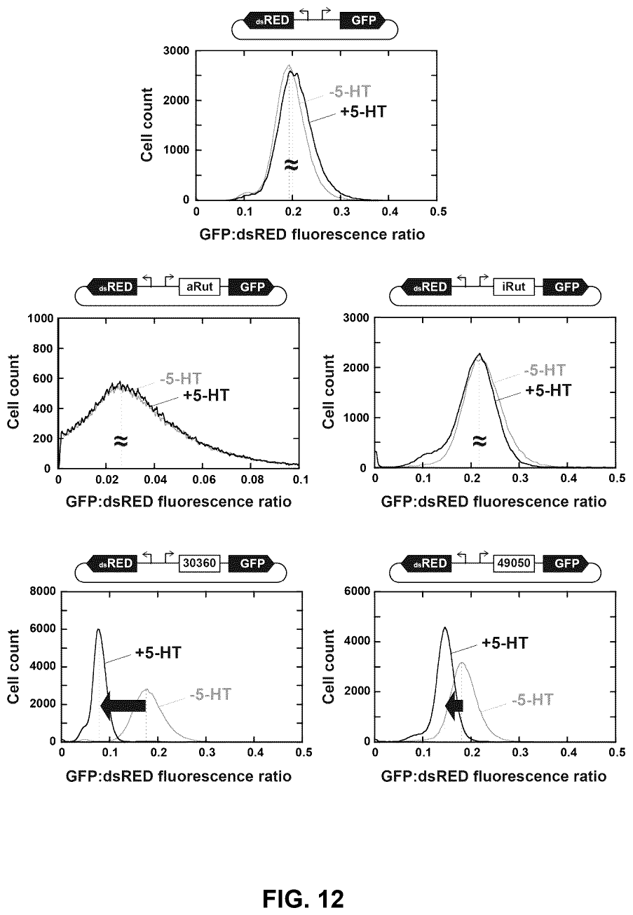

As used herein, the terms “modified SELEX process” and “helicase SELEX process” designate a process of selection of RNA or DNA aptamers, or riboswitches or desoxyriboswitches, as illustrated respectively in FIGS. 1, 3 and 4A. This helicase SELEX process is based on a step of functional selection of said aptamer/switch, wherein a library of nucleic acid duplexes is incubated in presence of an helicase of interest in an appropriate buffer for a sufficient time. The single-stranded nucleic acids that are released in the supernatant correspond to the separated strands from the duplexes, i.e. to the substrates of the helicase. These single strand nucleic acids are selectively recovered and amplified, according to a classical SELEX process.

Process for Selecting Aptamers Substrates of an Helicase

In a first aspect, the present invention concerns a process for selecting aptamers substrates of one helicase enzyme, comprising the implementation of a helicase SELEX process comprising several cycles, wherein each cycle comprises the following steps:
a) Providing a library of nucleic acid duplex constructs comprising one nucleic acid strand containing a random sequence of 10 to 100 nucleotides framed by fixed sequences at each end;
b) Incubation of said library with said helicase in appropriate conditions for the dissociation of certain duplex constructs by the helicase, resulting in release of the aptamers substrates of the helicase;
c) Isolation and amplification of said aptamers substrates of the helicase;
d) Creation of a novel library of nucleic acid duplex constructs enriched in duplex constructs comprising aptamers substrates of the helicase.

The helicase SELEX process can be implemented with any helicase enzyme known by the man skilled in the art. Numerous enzymes with an helicase function have been described in the literature, in all living organisms. For example, the human genome codes for 95 non-redundant helicases: 64 RNA helicases and 31 DNA helicases.

According to a specific embodiment of the helicase SELEX process, the helicase enzyme is Rho helicase. This enzyme, also designated as “Rho factor”, is a bacterial RNA-DNA helicase discovered in Escherichia coli in 1969. It is a homo-hexamer protein that recognizes and binds preferably to C-rich sites in the transcribed RNA, designed as “Rho utilization site (rut site)”. Once bound to RNA, Rho helicase unwinds RNA-DNA hybrids and releases RNA from a transcribing elongation complex, in an ATP-dependent process (23). In vitro, the Rho protein has a robust helicase activity that leads to the dissociation of RNA-DNA double strands.

According to another embodiment of the helicase SELEX process, the helicase enzyme is Upf1 enzyme. Human Upf1 is a RNA helicase involved in numerous DNA- and RNA-related processes, such as described in (24).

According to a specific embodiment of the process, the process for selecting aptamers substrates of one helicase enzyme is a process for selecting RNA aptamers. According to this implementation of the process, the nucleic acid strand containing a random sequence is a RNA single strand.

According to another specific embodiment of the process, the process for selecting aptamers substrates of one helicase enzyme is a process for selecting DNA aptamers. According to this implementation of the process, the nucleic acid strand containing a random sequence is a DNA single strand.

In this implementation of the process, the helicases are DNA helicases or promiscuous RNA helicases, able to act indifferently on DNA or RNA, such as for example Upf1.

DNA aptamers present the advantages to be more robust than RNA molecules, which are easily degraded by RNases.

The four essential steps of the helicase SELEX process are herein disclosed in details.

Step (a) consists of providing a library of nucleic acid duplex constructs, comprising one nucleic acid strand containing a random sequence of 10 to 100 nucleotides framed by fixed sequences at each end.

This library of nucleic acid duplex constructs contains double stranded nucleic acid molecules, consisting of:
RNA/DNA duplexes (case illustrated in FIGS. 1 and 4A)
RNA/RNA duplexes, or
DNA/DNA duplexes (case illustrated in FIG. 3).

These duplex constructs comprise two strands:
a first strand is a DNA or RNA strand containing a random sequence of 10 to 100 nucleotides framed by fixed sequences at each end. According to specific embodiments, this random sequence consists in 10, 20, 30, 40, 50, 60, 70, 80, 90 or 100 nucleotides.

This “random sequence” may be artificial or be issued from a genomic library. The term “random” indicates that the sequence of this nucleic acid fragment is unknown. In the present application, example 1 discloses a process applied to a synthetic library of artificial RNA fragments, and example 4 presents a process applied to a transcriptomic library of RNA fragments from the Escherichia coli genome.

In a preferred embodiment, all duplex constructs of the library present a random sequence having about the same length. This length is usually of 10 to 100 nucleotides.

This random sequence is framed by fixed, known sequences, at the 5′ and the 3′ extremities. For example, these framed sequences consist of 10, 20, 30, 40 or 50 nucleotides. These framed sequences usually comprise 10 to 20 nucleotides.

In a preferred embodiment, all duplex constructs of the library comprise the same fixed sequences at each end of the random sequences.
a second strand is a DNA strand, a RNA strand, or a 2′-alkyl-RNA strand that does not contain a random sequence.

In a preferred embodiment, this second nucleic acid strand of the duplex constructs hybridizes only with a portion of the first strand, preferably with a fixed sequence of the first strand.

In another embodiment, this second nucleic acid strand is biotinylated, allowing its further capture by streptavidin-bound beads.

Step (b) consists of the incubation of said library of duplex constructs with an helicase in appropriate conditions for the dissociation of certain duplex constructs by the helicase, resulting in release of the aptamers substrates of the helicase.

The “appropriate conditions of incubation” refer to the conditions of incubation allowing the “helicase reaction” to take place, i.e., designate the conditions in which the helicase enzyme is active. These appropriate conditions include incubation time, temperature, agitation, presence of cofactors such as ATP, all these conditions being well known by the man skilled in the art. For each helicase enzyme, the man skilled in the art will adapt said conditions of incubation in order to obtain an enzymatic reaction, corresponding to the dissociation of the duplex constructs that are substrates of said helicase.

For example, and as presented in the experimental section, the appropriate conditions for the dissociation of duplex constructs by the helicase Rho are the following:
The Rho helicase is used at a concentration comprised between 0.02 μM and 2 μM, for example about 0.6 μM; and/or
The helicase reaction is initiated by addition of MgCl2 (about 1 mM), ATP (about 1 mM), and 0 to 10 mM of the inducer ligand (for example, serotonin); and/or
The helicase reaction is performed for a time comprised between 20 seconds and 10 minutes, typically for about 2 minutes, at 37° C. under shacking, for example at about 300 rpm.

The release of aptamers substrates of the helicase corresponds to the dissociation of certain duplex constructs by the helicase; these dissociated single strands are released into the supernatant of the incubation medium, while the second strand is bound to beads, for example via a biotin-streptavidin interaction system.

Step (c) consists of the isolation and amplification of said aptamers substrates of the helicase. Isolation of aptamers present in the supernatant is usually performed by physical (e.g. filtration) or magnetic exclusion of the beads from the supernatant. Alternatively, the released single-strands can be separated from the unreactive duplexes by electrophoresis, on a SDS-PAGE gel for instance. In this specific case, bead- or surface-immobilization of the library of nucleic acid duplexes is not mandatory.

Amplification of the isolated single stranded nucleic acids may be carried out by any technique known by the man skilled in the art.

For example, for the amplification of isolated RNA molecules, a commonly used technique is RT-PCR (Reverse Transcription—Polymerase Chain Reaction).

Amplification by RT-PCR using a low fidelity polymerase such as the Taq polymerase can be advantageous: point mutations may appear during this step of amplification of the sequences. As a consequence, novel (yet closely related) sequences of aptamers may be generated and be tested in a further round of the process.

For the amplification of isolated DNA molecules, a commonly used technique is the asymmetric PCR, well known by the man skilled in the art.

Step (d) consists of the creation of a novel library of nucleic acid duplex constructs enriched in duplex constructs comprising aptamers substrates of the helicase, previously selected and amplified. This step is carried out according to the general knowledge of the man skilled in the art.

The process for selecting aptamer substrates of a specific helicase comprises several cycles, also designated as “rounds”, and in particular may comprise at least three cycles, at least five cycles, at least ten cycles, at least fifteen cycle or at least twenty cycles.

The process terminates when, after the step (c) of the last cycle, the step (d) is not performed and the selected aptamer substrates of the helicase are analyzed, preferentially are sequenced.

Specific Implementations of the Helicase SELEX Process for Selecting Aptamers

According to a specific implementation of the process of the invention, the nucleic acid duplex constructs are biotinylated and immobilized on streptavidin carrying beads, via the binding of the second strand of the duplex constructs, that does not contain a random sequence and is biotinylated.

According to another specific implementation of the process of the invention, steps (a) to (d) of the helicase SELEX process are automatically implemented by a robot, in particular by a liquid handling workstation.

FIG. 9 presents a diagram of the operations carried out by said robot. Automation allows the processing of up to 8 samples in parallel (diagram depicts the case for 4 samples). Legend of FIG. 9 presents the main steps of this implementation of the process.

Process for Selecting Switches Stimulating the Activity of an Helicase

The present invention is also related to a process for selecting switches stimulating the activity of one helicase enzyme in response to the presence of a specific inducer, comprising the implementation of a helicase SELEX process comprising several cycles, wherein each cycle comprises the following steps:
a) Providing a library of nucleic acid duplex constructs comprising one nucleic acid strand containing a random sequence of 10 to 100 nucleotides framed by fixed sequences at each end;
b1) Incubation of said library with said helicase and said specific inducer in appropriate conditions for the dissociation of certain duplex constructs by the helicase, resulting in release of the switches comprising the sequences that are substrates of the helicase in presence of said inducer; and/or
b2) incubation of said library with said helicase without any inducer for retention of switch-containing duplex constructs not dissociated by said helicase in absence of said inducer and elimination of duplex constructs dissociated by said helicase in absence of said inducer;
c) Isolation and amplification of said switches;
d) Creation of a novel library of nucleic acids duplex constructs enriched in duplex constructs comprising switches modulating the activity of the helicase in presence of a specific inducer,
and wherein at least one cycle comprises at least one step (b1).

This helicase SELEX process for the selection of switches can be implemented with any helicase enzyme known by the man skilled in the art. According to a specific embodiment of this helicase SELEX process, the helicase enzyme is Rho. According to another embodiment of the helicase SELEX process, the helicase enzyme is Upf1.

As previously specified, each switch consists in an aptamer domain and an expression platform domain. The random sequence selected and enriched with the Helicase SELEX process contains both domains.

According to a specific embodiment of the process, the selected switches are made of RNA (riboswitches). According to this implementation of the process, the nucleic acid strand containing a random sequence is a RNA single strand.

According to another specific embodiment of the process, the selected switches are made of DNA (desoxyriboswitches). According to this implementation of the process, the nucleic acid strand containing a random sequence is a DNA single strand.

This process is illustrated in FIGS. 4A and 4B (illustrating steps b1 and b2). It presents several common elements with the process of selection of RNA or DNA aptamers as presented above.

In particular, the nucleic acid duplex constructs may be biotinylated and immobilized on streptavidin carrying beads, via the binding of the second strand of the duplex constructs, that does not contain a random sequence and is biotinylated.

Further, steps (a) (c) and (d) are common with the process of selection of aptamers, and are not detailed again.

The main difference between processes of selection of aptamers and switches is the use of an inducer that activates the switch, which in turn stimulates the helicase activity in the selection step (b1).

This inducer may be a natural inducer, in particular serotonin, or a synthetic inducer.

The inducer will be used in an efficient quantity. In particular, the man skilled in the art will use its general knowledge for adjusting the concentration of the inducer for improving the selection pressure of the selection process.

Steps (b1) and (b2) are Hereby Disclosed in Details.

Both steps are illustrated in FIG. 4B.

Step (b1) consists of the incubation of a library of nucleic acid duplexes with a helicase and a specific inducer compound, in appropriate conditions for the dissociation of certain duplex constructs of the library by the helicase, resulting in release of the switches comprising the sequences that are substrates of the helicase in presence of said inducer. This step is designated as a step of “selection” in presence of the inducer compound, and allows the selection of switches sensitive to the inducer, present in an efficient quantity as determined by the man skilled in the art.

Step (b2) consists of the incubation of said library with said helicase without any inducer for inducing the retention of switch-containing duplex constructs not dissociated by the helicase in absence of the inducer compound, and elimination of duplex constructs dissociated by the helicase even in absence of said inducer. This step is designated as a step of “counter-selection” since it allows the elimination of inducer-independent aptamers of the helicase from the library.

The process of selection of the invention comprises several cycles, also designated as “rounds”, and in particular may comprise at least three cycles, at least five cycles, at least ten cycles, at least fifteen cycle or at least twenty cycles.

The process of selection of the invention comprises at least one cycle comprising at least one step (b1) of selection.

In a specific implementation of the process, step (b1) is performed during at least one cycle, at least two cycles, or at least three cycles of the process. In another embodiment, step (b1) is performed during at least 1/10 of the cycles, preferably during ¼ of the cycles, and more preferably during about half the cycles of the process.

In a specific implementation of the process, step (b2) is performed during at least one cycle, at least two cycles, or at least three cycles of the process. In another embodiment, step (b2) is performed during at 1/10 of the cycles, preferably during ¼ of the cycles.

In a specific implementation of the process, steps (b2) and (b1) are sequentially performed during a single cycle of the process. The duplex library is first incubated in the presence of the helicase and in absence of the inducer; the reaction supernatant containing the inducer-independent sequences is discarded (step b2). The beads bearing the remaining duplexes are recovered and then incubated with a new batch of the helicase in the presence of the inducer; the newly released, inducer-dependent sequences are recovered from the supernatant (step b1).

In table 5 in the experimental section, this alternative (b2)+(b1) step is designated as a “mixed” step. In a specific implementation of the process, mixed step (b2) and (b1) is performed during at least one cycle, at least two cycles, or at least three cycles of the process.

In a specific implementation of the process, successive cycles comprising a step (b2) and/or a step (b1) are carried out in any order throughout the process.

Table 5 in the examples section illustrates, as an example, a process comprising 21 rounds/cycles, wherein:
step (b1) of selection is carried out during the 10 first cycles;
step (b2) of counter-selection is carried out for three cycles;
a mixed step (b1) and (b2) is carried out during the last 8 cycles.

The helicase SELEX process for selecting switches comprises several cycles, also designated as “rounds”, and in particular may comprise at least three cycles, at least five cycles, at least ten cycles, at least fifteen cycles or at least twenty cycles.

The helicase SELEX process of switches terminates after the step (c) of the last cycle, the step (d) being not performed, and the selected/enriched switches are analyzed, preferentially are sequenced.

According to a specific implementation of the process of the invention, steps (a) to (d) of the helicase SELEX process are automatically implemented by a robot, in particular by a liquid handling workstation.

FIG. 9 presents a diagram of the operations carried out by said robot. Automation allows the processing of up to 8 samples in parallel (diagram depicts the case for 4 samples). Legend of FIG. 9 presents the main steps of this implementation of the process.

Aptamers and Switches Such as Obtained

The present invention also concerns isolated aptamers, substrates of a helicase, obtained by the process as described above.

These aptamers are qualified as being “synthetic” or “artificial” if they have been selected from a library of synthetic nucleic acid fragments (see example 1). They are qualified as being “natural” in the case they have been isolated from a library of natural nucleic acid fragments (see example 4, FIGS. 10A and 10B).

The present invention also concerns isolated switches (riboswitch or desoxyriboswitch), modulating the activity of a helicase enzyme in response to the presence of a specific inducer, obtained by the process as described above.

As specified above, said inducer may be natural or synthetic.

These switches may be qualified as being “synthetic” or “artificial” if they have been selected from a library of synthetic switches (see example 2). They are qualified as being “natural” in the case of they have been isolated from a library of natural nucleic acid fragments.

Up to now, natural forms of desoxyriboswitches have never been described. The process according to the invention allows, for the first time, the generation of synthetic desoxyriboswitches.

Uses of Selected Switches

The present invention also concerns a genetic construction comprising one switch selected with the process described above, and an expression cassette.

In this genetic construction, the switch is sensitive to a specific inducer. The expression cassette is under the control of said switch, which induces the expression of said expression cassette when it is activated.

An expression cassette is composed of one or more genes and the sequences controlling their expression.

Said expression cassette may comprise any type of gene of interest: a gene coding for a therapeutic protein, coding for a toxic protein, coding for a reporter protein, or any other protein of interest.

According to a specific embodiment, the gene of interest encodes a reporter protein such as a fluorescent protein. The genetic construction is, in this case, a reporter system sensitive to the presence of a specific inducer compound: the reporter protein will be either repressed or expressed only in presence of said inducer. For instance, recruitment of the Rho helicase on the mRNA by the switch will lead to Rho-dependent termination of transcription and silencing of the reporter. Similarly, recruitment of Upf1 on the mRNA could trigger mRNA decay and reporter silencing whereas recruitment of a helicase involved in translation initiation could trigger expression of the reporter.

Desoxyriboswitches made of DNA, since they are less fragile than RNA molecules, may be used as direct biosensors. As illustrated in FIG. 11, sensing of the presence of a given analyte (i.e., the inducer used for selection of the switch by Helicase SELEX) in a sample of interest (e.g. clinical or environmental sample) may be achieved with a switch-containing duplex labeled with a fluorophore-quencher pair, the dye and quencher being attached to distinct strands of the duplex. Physical separation of the dye and quencher moieties upon duplex unwinding by the helicase results in a fluorescence increase that is used to monitor the presence of the analyte in the analyzed sample. Samples of interest, in particular, clinical and environmental samples are often complex media wherein RNA is at a greater risk of degradation than is DNA (due to the abundance of environmental RNases, for instance). Degradation of a duplex strand may artificially release the structural proximity imposed on the dye-quencher pair and yield false-positive fluorescence.

The present invention also concerns a method for detecting a compound of interest, using the reporter system as described above, wherein the switch included in the genetic construction is sensitive to the presence of said compound of interest. This method of detection may be implemented in vivo and in vitro.

EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Material & Methods

Materials

Unless specified otherwise, chemicals and enzymes were purchased from Sigma-Aldrich and New England Biolabs, respectively. Nucleoside triphosphates and radionucleotides were purchased from GE-Healthcare and PerkinElmer, respectively. Synthetic oligonucleotides were obtained from Eurogentec and are listed in Table 1. The Rho protein was prepared and purified as described previously (3). Rho concentration is expressed in hexamers throughout.

TABLE 1
Oligonucleotides

| Designation of | | SEQ |
|---|---|---|
| the sequence | Nucleotidic sequence | ID No. |
| Starting library of | 5′GGGAGACCGGCCAGC-(N₅₀)- | 1 |
| DNA, further | CGATGGTATCAGATCTGGATCCTCGAGAAGCTGC | |
| transcribed to | | |
| RNA | | |
| (for pilot | | |
| ′aptamer′ | | |
| experiment) | | |
| Starting library of | 5′GGGAGACCGGCCAGC-(N₈₀)- | 2 |
| DNA, further | CGATGGTATCAGATCTGGATCCTCGAGAAGCTGC | |
| transcribed to | | |
| RNA | | |
| (for pilot ′switch′ | | |
| experiment) | | |
| FWD | 5′CGAAATTAATACGACTCACTATAGGGAGACCGGCCAGC | 3 |
| REV | 5′CGATGAATTCGAGCTCGGTACCCGCAGCTTCTCGAGGATCCAGAT | 4 |
| | CTGATACCATCG | |
| SEL | 5′biotin-TTTTTTTTTTCGATGAATTCGAGCTCGGTACCCGCAGCTTC | 5 |
| | TCGAGGATCCAGATCTGATACCATCG | |
| TRAP | 5′CGATGGTATCAGATCTGGATCCTCGAGAAGCTGCGGGTACCGAG | 6 |
| | CTCGAATTCATCG | |
| LESS-OLN | 5′GGGAGACCGGCCAGCCGATGGTATCAGATCTGG | 7 |
| FORWA | 5′CGGCTCGTATAATGTGTGGAATTGTGAGCGGATAACAATTTGGGA | 8 |
| | GACCGGCCAGC | |
| FORWB | 5′- | 9 |
| | GCGGCCGCACTCGAGGAGCTGTTGACAATTAATCATCGGCTCGTAT | |
| | AATGTGT | |
| FACS-REV | 5′ACTGTCTTACACACCGGTAAGACAGCCCAGATCTGATACCATCG | 10 |
| C1-OLN | 5′GGGAGACCGGCCAGCCCCATGTATCGTCGAGGGCAGTTCTTGGA | 11 |
| | TCCTCTGTAAGAGATTACGGTTATCTCCGTATGAAACAGTTGTTTAC | |
| | CCTGCGATGGTATCAGATCTGG | |

^{a}: Constant upstream and downstream sequences are shown in italic (upstream) and bold characters (downstream). Sequence of the T7 promoter is underlined.

Nucleic acid quantitation was performed by standard UV spectrophotometry using a Nanodrop spectrophotometer and, for analytical assays, was verified with Quant-iT PicoGreen (DNA) and RiboGreen (RNA) fluorescence detection kits (Thermo-Fisher Scientific).

Culture Media

We used lysogeny broth medium (LB medium; Sigma-Aldrich) for standard Escherichia coli cultures (e.g. in cloning and plasmid preparation procedures) and a tryptophan-less version of the Neidhardt supplemented MOPS defined medium (4) with 0.2% glucose (hereafter named Wless medium) for in vivo characterization of serotonin-dependent candidates. Antibiotic carbenicillin was used at a concentration of 100 lag/mL in all instances.

Preparation of the Starting RNA:DNA Duplex Library

A single-stranded DNA (ssDNA) library was purchased from Eurogentec. Each library member contained 50 (‘aptamer’ experiment) or 80 (‘switch’ experiment) randomized nucleotides flanked by two primer-binding regions for PCR (Table 1). About 1 nmole of the starting ssDNA library was converted in 6 cycles of PCR amplification (95° C. for 1 min, 50° C. for 1 min, 72° C. for 1 min) into a library of double-stranded DNA (dsDNA) templates for T7 transcription. The initial PCR mixture contained 0.4 μM of ssDNA library, 4 μM of FWD and REV primers, 0.2 mM dNTPs (each), and 50 U of Taq DNA polymerase in 2 mL of Taq buffer (10 mM Tris-Cl, pH 8.5, 50 mM KCl, 1.5 mM MgCl₂, 0.1% Triton X-100) and was split in ten 0.5 mL microtubes for amplification. The resulting dsDNA library was purified with the GeneJET PCR purification kit (Thermo Fisher Scientific) and used directly for in vitro transcription with T7 RNA polymerase. The transcription reaction was performed as described previously (3), except that the scaled-up reaction volume (5 mL) was split in ten parallel microtubes for incubation at 37° C. Following concentration by ethanol precipitation, the resulting single-stranded RNA (ssRNA) library was purified on custom-made, 1 mL Sephadex G50 spin columns, phenol extracted and ethanol precipitated. Alternatively, transcription crude was incubated with RNase-free DNase I and purified with a RNA clean and concentrator kit (Zymo research) in a procedure more suitable for automation on a liquid handling workstation. Transcripts were resuspended and stored in M₁₀E₁ (10 mM MOPS, 1 mM EDTA, pH 6.5) buffer at −20° C. To allow monitoring of reaction species during Helicase-SELEX, a fraction of the ssRNA library (˜10 pmoles) was dephopshorylated with calf intestine phosphatase and 32P-labeled with gamma[P32]-ATP and T7 polynucleotide kinase, as described (3). The 32P-labeled ssRNA and 2 nmoles of unlabeled ssRNA library were then mixed in hybridization buffer (150 mM potassium acetate, 20 mM HEPES pH 7.5, 0.1 mM EDTA) before addition of 1.1 molar equivalent of 5′-biotinylated SEL oligonucleotide. The mixture was incubated at 70° C. for 2 min and then cooled to room temperature for 15 min before use in Helicase-SELEX.

Helicase-SELEX Assays

The library of RNA:DNA duplexes was immobilized on streptavidin-coated magnetic beads (Dynabeads, Thermo Fisher Scientific) following a protocol described previously (5). Briefly, beads (˜1 μL of bead slurry per pmole of RNA:DNA duplexes) were washed with BW buffer (1M KCl, 5 mM Tris-Cl, pH 7.5, 0.5 mM EDTA) before addition to the crude mix of RNA:DNA hybrids (from section above) and incubation for 1 h at room temperature. Beads were then magnetically separated from supernatant on a MagRack (GE Healthcare), washed first with BW buffer and then with helicase buffer (hybridization buffer supplemented with 0.1 mg/mL of acetylated BSA) to remove unbound, non-biotinylated RNA/DNA species. A total of ˜1 nmole (6×10¹⁴ molecules) of bead-immobilized RNA:DNA substrates were used in the first selection round (R1) while lower amounts were used in subsequent rounds (450 to 50 pmoles). The bead-affixed substrates (0.2 μM, final concentration) were incubated with Rho (0.6 μM, final concentration) in helicase buffer for 10 min at 37° C. Then, the helicase reaction was initiated by addition of 1 mM MgCl₂, 1 mM ATP, and 0-10 mM inducer ligand (e.g. serotonin) and incubated for 2 min at 37° C. under shacking at 300 rpm to homogenize the bead suspension. Supernatant containing the released ssRNA strands was magnetically separated from the beads on the MagRack stand. For selection rounds, the supernatant was kept for subsequent steps; for counter-selection rounds, the beads were either washed thrice with helicase buffer and reused directly in a new reaction with the Rho helicase or heat-denatured in M₁₀E₁ buffer and the supernatant kept for subsequent steps.

The ssRNA strands present in supernatant were extracted with phenol, purified on a G50 spin column, and precipitated with ethanol (or these steps were replaced by purification with a RNA clean & concentrator kit from Zymo Research). Then, they were reverse transcribed with the REV primer (1.2 molar equivalent) and Superscript III reverse transcriptase (Invitrogen; 2 U per pmole of ssRNA) in the First Strand buffer supplied with the enzyme. Reaction mixtures were incubated for 1 h at 50° C. and then for 15 min at 70° C. The ssDNA products were amplified by PCR (12 cycles) with the Taq DNA polymerase using the FWD and REV primers (0.6 μM, final concentrations) to generate the dsDNA template library for the next round of T7 transcription, assembly of the RNA:DNA duplex library, and functional selection by Helicase-SELEX (performed as described in section above).

Automation of the above ‘Helicase-SELEX’ procedure was performed in a 96-well multiplate format on a TECAN Evo150 robotic platform equipped with AirLiHa and Roma arms and dedicated temperature-controlled INHECO modules for storage of reactants (CPAC modules), shaking (Thermoshake module), and thermocycling (ODTC module) (FIG. 9). Magnetic separation of reaction products was performed on a 96-well Magnum FLX magnet plate (Alpaqua) while purifications by phenol extraction, G50 spin column, and ethanol precipitation steps were replaced by vacuum-based solid-phase extraction on a TECAN Te-Vacs module equipped with a custom-made adapter for Clean & Concentrator columns (Zymo Research). The Evo150 robotic platform was operated with custom-made Evoware scripts allowing full automation of the ‘Helicase-SELEX’ cycle (with an optional intermediate counterselection step), reaction times as short as 20 s, and optional handling of several samples in parallel.

Variations in Helicase-SELEX conditions (library size, concentrations of reactants, incubation time, etc.) were introduced in some rounds to limit biases and increase selection stringency. Notably, an alternative manual selection approach was implemented and tested to avoid potential selection biases or entropic artefacts due to bead immobilization (6,7). In this case, the library of RNA:DNA duplexes was prepared with the non-biotinylated REV oligonucleotide and was purified by native 6% polyacrylamide gel electrophoresis (PAGE) as described previously (3). The purified RNA:DNA duplexes (80 nM) were mixed with Rho (80 or 320 nM) and serotonin (0, 1, or 10 mM) in helicase buffer and incubated for 10 min at 37° C. The helicase reaction was initiated by addition of a mix containing MgCl₂ and ATP (1 mM, final concentrations) as well as oligonucleotide TRAP (800 nM, final concentration), which is complementary to the REV oligonucleotide. Reaction was quenched by addition of SDS (2% final concentration) and EDTA (80 mM final concentration) after 0.5 to 20 min of incubation at 37° C. The pool of released RNA strands (selection scheme) or unreactive RNA:DNA duplexes (counterselection scheme) was purified on a 9% PAGE gel containing 0.5% SDS (8). Reverse transcription and PCR amplification to generate the dsDNA template library for the next round of Helicase-SELEX were performed as described above.

Analysis of Sequence Libraries

The dsDNA pools obtained after rounds 8, 13, 15, and 21 were analyzed by 2×150 base paired-end sequencing on a Miseq Illumina instrument at the IMAGIF sequencing platform of CNRS (Gif-sur-Yvette, France). Starting, blunt-ended dsDNA pools (˜2.5 μg each) were processed by IMAGIF using standard Miseq procedures and the MiSeq reagent kit v2 (Illumina). Samples were supplemented with DNA from coliphage phiX174 to mitigate the potentially low sequence diversity of Helicase-SELEX sequence pools (9). Due to the large size of the DNA fragments (175 bp plus adapters), the forward and reverse reads (R1 and R2 reads in standard Illumina nomenclature; distinct from our R1 and R2 libraries of aptamer/switch sequences) were concatenated rather than processed through paired-end assembly. The upstream and downstream constant sequences (Table 1) were used to select and orient the reads in the same, top strand direction (RNA strand orientation in the hybrid duplexes). Multiplexed sequencing of the DNA pools resulted in ˜10×10⁶ correctly oriented reads per pool after quality control filtering, adapter and constant sequence trimming, and elimination of coliphage phiX174 sequences.

Construction of Dual Reporter Plasmids

Control plasmid pFACS-aRut-mgtA-Tac1 (FIG. 7) was obtained by subcloning a synthetic DNA fragment (purchased from Genscript) containing the dsRED-Express2 and sfGFP reporter genes under control of divergent Ptac promoters between the AatII and AvrII sites of plasmid pZE12luc (kindly provided by Pr. Bujard, University of Heidelberg) (10). The synthetic DNA fragment also contains a strong, artificial rut sequence (aRut) (11,12) and the 135-251 region of the mgtA leader of Salmonella enterica (13) between the sfGFP reporter and its driving promoter, as well as intrinsic terminators TO and T1 respectively downstream from the dsRED-Express2 and sfGFP reporter genes (FIG. 7).

TABLE 2
Control Sequences

| | | SEQ ID |
|---|---|---|
| Relevant sequences | | No. |
| aRut (positive control: | 5′ACUUCUCCUCUGUCUCCUUCUUCCUUCUCCUC | 12 |
| Rho inducer) | UGUCUCCUUCUUCCUCGACC | |
| iRut (negative control: | 5′GGUCGAGGAAGAAGGAGACAGAGGAGAAGGA | 13 |
| inverse sequence of aRut) | AGAAGGAGACAGAGGAGAAGU | |

Control plasmid pFACS-RutLess-mgtA-Tac1 was obtained by subcloning a PCR-amplified DNA fragment devoid of aRut sequence into the XhoI and BglII restriction sites of plasmid pFACS-aRut-mgtA-Tac1 (FIG. 7). The fragment was obtained by PCR amplification of oligonucleotide LESS-OLN, first with primers FORWA and FACS-REV and then with primers FORWB and FACS-REV. Control plasmid pFACS-iRut-mgtA-Tac1 bearing the inactive reverse complement sequence of aRut sequence (FIG. 7) and dual reporter plasmids containing the R21-49050 and R21-30360 sequences instead of the aRut sequence of pFACS-aRut-mgtA-Tac1 (FIG. 7) were constructed by similar subcloning and PCR procedures. Plasmid variants devoid of the mgtA leader region (tsp-less plasmid series) were prepared by deletion of the BglII-XbaI fragment and filled-in ligation of the corresponding parent plasmid. Plasmid sequences were verified by standard DNA sequencing (Genoscreen, Lille, France).

Construction of the R13 Dual Reporter Plasmid Library and Selection of Candidate Sequences

Sequences from the DNA template library (˜2 pmoles) obtained after round 13 of Helicase-SELEX were equipped with an upstream pTac promoter in two successive PCR reactions (6 cycles each), first with primers FORWA and FACS-REV and then with primers FORWB and FACS-REV (see table 1). The resulting DNA fragment library was subcloned into the XhoI and BglII sites of plasmid pFACS-aRut-mgtA-Tac1 (FIG. 7). Ligation products were transformed into DH5α cells and incubated overnight at 37° C. in LB medium supplemented with carbenicillin. Approximately 109 transformants were obtained (estimation based on plating diluted library aliquots on LB-carbenicillin agar plates). Cells were pelleted by centrifugation and stored at −80° C. in 20% glycerol until use.

Cells harboring the R13 dual reporter plasmid library were plated on LB-carbenicillin agar plates and incubated overnight at 37° C. Plates were imaged with a Typhoon FLA-9500 imager (GE Healthcare) and the sfGFP:dsRED-Express2 fluorescence ratios of well isolated colonies were determined with ImageQuant TL software (ratios ranged between −0.1 and ˜5 with this method). Colonies displaying high (>3) sfGFP:dsRED-Express2 ratios were picked randomly and used to inoculate 1 mL aliquots of Wless medium supplemented with carbenicillin (Wless-carbenicillin medium). The resulting reporter plasmids harboring R13 sequences instead of the aRut sequence of the pFACS-aRut-mgtA-Tac1 plasmid (FIG. 7) were purified from overnight cultures with the Nucleospin plasmid kit (Macherey-Nagel) and Sanger-sequenced by Genoscreen (Lille, France).

Analysis of In Vivo Reporter Fluorescence by Flow Cytometry

DH5α cells harboring the control, R13 or R21 reporter plasmids described above were grown overnight at 37° C. in Wless-carbenicillin medium. The cultures were diluted 100-fold in 1 mL of fresh Wless-carbenicillin medium containing 0 or 10 mM serotonin and were incubated for 2 h at 37° C. under shacking at 230 rpm (as control, plasmid-less DH5alpha cells were similarly cultured in Wless medium). Cells were pelleted by centrifugation, washed with 1 mL of phosphate-buffered saline (PBS), and suspended in 1 mL of PBS. Samples were analyzed by flow cytometry with an LSRFortessa X20 cell analyzer (Becton Dickinson) equipped with 488 nm and 570 nm lasers and 530/30 nm and 586/15 nm band-path emission filters for sfGFP and dsREDexpress2, respectively. The sfGFP and dsRED-Express2 fluorescence of ˜100,000 cells was measured for each sample. Data were analyzed with FACSDiva (Becton Dickinson) and Flowing Software 2.5.1 (http://flowingsoftware.btk.fi) using gating based on forward scatter intensity and background fluorescence of plasmid-less cells, as recommended (14).

Duplex Unwinding Kinetics

Duplex substrates were prepared by hybridizing ³²P-labeled transcripts (from a given round library or corresponding to a single winner sequence) with the REV oligonucleotide and were purified by native 6% PAGE (3). Helicase kinetics were determined with the purified ³²P-labeled RNA:DNA duplexes as described previously (12) (15), with minor modifications. Briefly, duplexes (5 nM) were mixed with Rho hexamers (20 nM) in helicase buffer (supplemented with the indicated concentration of inducer ligand, e.g. serotonin) and incubated for 3 min at 37° C. Then, 1 mM MgCl₂, 1 mM ATP, and 400 nM oligo TRAP were added to the helicase mixture before further incubation at 37° C. Reaction aliquots were taken at various times and mixed with two volumes of quench buffer (10 mM EDTA, 1.5% SDS, 300 mM sodium acetate, 6% Ficoll-400) before being loaded on 9% PAGE gels that contained 1×TBE and 0.5% SDS. Detection and quantification of gel bands were performed by phosphorimaging with a Typhoon FLA-9500 imager, as described (16).

RNA Binding Assay

Equilibrium Rho-RNA dissociation constants were determined with an electrophoretic mobility shift assay (EMSA) adapted from previous work (17). Briefly, 0.1 nM of ³²P-labeled RNA-DNA hybrid substrate were mixed with increasing amounts of Rho in binding buffer (20 mM HEPES, pH 7.5, 0.5 mM EDTA, 0.5 mM DTT, 150 mM potassium acetate, 30 μg/ml tRNA, and 20 μg/ml BSA) in the presence of 0 or 10 mM serotonin. After incubation for 15 min at 37° C., the samples were supplemented with 4% Ficoll-400 and 0.1% Triton X-100 and analyzed by PAGE on native 6% polyacrylamide gels containing 0.1% Triton X-100. The fractions of free and Rho-bound RNA were then determined by phosphorimaging of the gels with a Typhoon FLA-9500 imager.

Results

### Example 1. Selection of Aptamers Substrates of Rho Enzyme with a Helicase SELEX Process According to the Invention

A library of RNA-DNA duplexes containing a region of randomized sequence of 50 nucleotides was prepared as detailed in the material and methods. The randomized region is flanked by fixed sequences that allow the assembly of the RNA-DNA duplexes and the amplification of the selection products by RT-PCR (with the FWD and REV primers, sequences are presented in table 1).

RNA-DNA duplexes are biotinylated to be immobilized on streptavidin-coated beads (typically Dynabead M-280 or Dynabeads My One T1 magnetic beads), to allow the selection. The immobilized duplexes are then incubated in the presence of Rho and ATP to initiate the helicase reaction. The “reactive” duplexes are dissociated by the helicase activity of Rho and the RNA strands containing the “winning” sequences (Rut sites) are easily isolated in the supernatant (since inactive duplexes remain attached to the beads) and then amplified by RT-PCR.

The in vitro transcription of the DNA matrices thus generated is used to prepare a new library of RNA-DNA duplexes for a new Helicase-SELEX cycle, and an iterative enrichment in winning sequences.

When the enrichment is considered sufficient, after several Helicase-SELEX cycles, the “winning” sequences are determined by sequencing of the DNA template library. These sequences can then be evaluated individually.

TABLE 3
Summary of conditions used during Helicase-SELEX rounds for selection of aptamers

| | Duplex | | | | | | | |
|---|---|---|---|---|---|---|---|---|
| | library | | | | | RNA:DNA | | reaction |
| | size | Selection | Selection | Reaction | Temperature | duplex | Rho | time |
| Round | (pmol) | yield (%) | mode | scheme^{a} | (° C.) | (μM) | (μM) | (min) |
| 1 | 1222 | 10 | beads | selection | 25 | 0.2 | 0.6 | 15 |
| 2 | 417 | 5 | beads | selection | 25 | 0.2 | 0.6 | 5 |
| 3 | 440 | 2 | beads | selection | 25 | 0.2 | 0.6 | 15 |
| 4 | 446 | 5 | beads | selection | 25 | 0.2 | 0.6 | 15 |
| 5 | 414 | 22 | beads | selection | 25 | 0.2 | 0.6 | 15 |
| 6 | 393 | 10 | beads | selection | 25 | 0.2 | 0.6 | 0.5 |
| 7 | 372 | 12 | beads | selection | 25 | 0.2 | 0.6 | 0.5 |

^{a}Reactive ssRNA strands are recovered from the supernatant.

After each cycle, a library of RNA-DNA duplexes is obtained and designated Rx with x=number of cycles.

A library of highly reactive sequences has been obtained after only 7 Helicase-SELEX cycles (R7). More than 50% of the DNA-RNA duplexes of the R7 library are dissociated in a few minutes in the presence of Rho and ATP, whereas the starting duplexes of the original library “R0” presented a negligible activity, as shown in FIG. 2. The “unwound fraction” corresponds to the fraction of RNA aptamers dissociated from the DNA by the Rho activity.

The two most abundant aptamers of the R7 library (1-386 and 2-355) have been sequenced and their sensitivity to Rho helicase has been confirmed (data not shown).

TABLE 4
Sequences of the two most abundant aptamers of the R7 library
(1-386 and 2-355)
The constant ssRNA sequence present in the duplexes upstream from
the variable sequence (as shown in FIG. 5A) is underlined

| | | SEQ ID |
|---|---|---|
| | | No. |
| 1-386 | 5′GGGAGACCGGCCAGCTGUUUGGCUGGAAUCCGUUUCUUG | 14 |
| | GCUCAUUUCUGGGUCAAAUUCUCUGU | |
| 2-355 | 5′GGGAGACCGGCCAGCUUGGGUCGGCCAAUCCCGUGUCUU | 15 |
| | GCAGUAUUUCCACUGCGACCUUCCUA | |

### Example 2. Selection of Riboswitches Stimulating the Activity of Rho Helicase in Presence of Serotonin, with a Helicase SELEX Process According to the Invention

FIGS. 4A and 4B illustrate the principle of the Helicase SELEX process with or without inducer (4A) and the principles of selection and counter-selection for inducer activated-switches (4B).

A library of RNA-DNA duplexes containing a region of randomized sequence of 80 nucleotides was prepared as detailed in the material and methods. The randomized region is flanked by fixed sequences that allow the assembly of the RNA-DNA duplexes and the amplification of the selection products by RT-PCR (with the FWD and REV primers as presented in table 1).

After each cycle, a library of RNA-DNA duplexes is obtained and designated Rx with x=number of cycles.

The library is incubated in the presence of Rho, ATP and serotonin (5-HT, 1 or 10 mM) to initiate the helicase reaction. As presented at the top of FIG. 5B, the “reactive” duplexes are dissociated by the helicase activity of Rho and the RNA strands containing the “winning” sequences are isolated in the supernatant and then amplified by RT-PCR.

As the cycles progress, the quantity of duplexes susceptible to the helicase activity in presence of serotonin increases, as is shown in FIG. 5.

TABLE 5
Summary of conditions used during Helicase-SELEX rounds

| | Duplex | | | | | | | |
|---|---|---|---|---|---|---|---|---|
| | library | | | | | RNA:DNA | | reaction |
| | size | Selection | Selection | | Serotonin | duplex | Rho | time |
| Round | (pmol) | yield (%) | mode | Reaction scheme^{a} | (mM) | (μM) | (μM) | (min) |
| 1 | 1000 | 16 | beads | selection | 1 | 0.2 | 0.6 | 2 |
| 2 | 450 | 14 | beads | selection | 10 | 0.2 | 0.6 | 0.5 |
| 3 | 450 | 18 | beads | selection | 10 | 0.2 | 0.6 | 0.5 |
| 4 | 50 | n.d. | PAGE | selection | 10 | 0.08 | 0.32 | 15 |
| 5 | 50 | n.d. | PAGE | selection | 10 | 0.08 | 0.32 | 2 |
| 6 | 50 | n.d. | PAGE | selection | 10 | 0.08 | 0.32 | 0.5 |
| 7 | 50 | n.d. | PAGE | selection | 10 | 0.08 | 0.08 | 0.5 |
| 8 | 50 | n.d. | PAGE | selection | 10 | 0.08 | 0.08 | 0.5 |
| 9 | 50 | n.d. | PAGE | selection | 10 | 0.08 | 0.08 | 0.5 |
| 10 | 50 | n.d. | PAGE | selection | 10 | 0.08 | 0.08 | 0.5 |
| 11 | 50 | n.d. | PAGE | counterselection | 0 | 0.08 | 0.08 | 10 |
| 12 | 50 | n.d. | PAGE | counterselection | 0 | 0.08 | 0.08 | 15 |
| 13 | 50 | n.d. | PAGE | counterselection | 0 | 0.08 | 0.08 | 20 |
| 14 | 100 | 1 | beads | mixed | 0 then 1 | 0.08 | 0.08 | 20 then 2 |
| 15 | 100 | 1 | beads | mixed | 0 then 1 | 0.08 | 0.08 | 20 then 1 |
| 16 | 100 | 8 | beads | mixed | 0 then 1 | 0.08 | 0.08 | 20 then 1 |
| 17 | 100 | 7 | beads | mixed | 0 then 1 | 0.08 | 0.08 | 20 then 1 |
| 18 | 100 | 3 | beads | mixed | 0 then 1 | 0.08 | 0.08 | 20 then 1 |
| 19 | 100 | 1 | beads | mixed | 0 then 1 | 0.08 | 0.08 | 20 then 1 |
| 20 | 100 | 3 | beads | mixed | 0 then 1 | 0.08 | 0.08 | 20 then 1 |
| 21 | 100 | 3 | beads | mixed | 0 then 1 | 0.08 | 0.08 | 20 then 1 |

^{a}Reactive ssRNA strands or unreactive RNA:DNA duplexes are recovered during ‘selection’ (with serotonin) and ‘counterselection’ (without serotonin) reactions, respectively. In mixed schemes, a ‘counterselection’ is first performed in absence of serotonin; beads bearing the unreactive duplexes are then separated from the supernatant and directly used in a ‘selection’ reaction in presence of serotonin.

As shown in FIG. 6A, the library R13 (obtained after 13 cycles), is the most sensitive to the presence of 10 mM serotonin, whereas libraries R20 and R21 (obtained after additional cycles) are the most sensitive to lower concentrations of the inducer.

In FIG. 6B, the R13 duplex library is tested: time courses (0-40 min) of Rho helicase reactions performed with the 32P-labeled R13 duplex library in the presence of 0 (−5-HT) or 10 mM (+5-HT) serotonin were monitored by PAGE (gel images shown on top of the figure) and phosphorimaging. Quantitation of the fraction of duplexes unwound by Rho as a function of time shows that the R13 library is highly sensitive to the presence of serotonin.

The libraries R8, R13, R15 and R21 have been sequenced (Illumina, TruSeq genomic, 150 paired-end). Results are as follow:

TABLE 6
Results of the HELICASE process of selection of riboswitches

| Duplex library | R8 | R13 | R15 | R21 |
|---|---|---|---|---|
| Number of distinct | 8,627,858 | 8,626,305 | 4,209,973 | 3,155,933 |
| sequences (>50 nt) | | | | |
| Frequency of the most | 6 | 6 | 134 | 49050 |
| abundant sequence | | | | |

Enrichment is late but notable. The R21 library contains many clusters of close sequences. For example, the most abundant R21 sequence is R21-49050. The table 7 below presents four enriched sequences.

Sequences R13-C21 and R13-C37 were obtained by colony picking and Sanger sequencing as described in the methods section above. Sequences R21-49050 and R21-30360 are the most abundant sequences of the R21 library, as determined by NGS sequencing. The constant ssRNA sequence present in the duplexes upstream from the variable sequence (as shown in FIG. 5A) is underlined.

TABLE 7
Sequences of four representative sequences obtained after 13 and 21
rounds (R13 and R21)

| Designation of | | SEQ ID |
|---|---|---|
| sequences | | No. |
| R13-C1 | 5′GGGAGACCGGCCAGCCCCAUGUAUCGUCGAGGGCAGUUC | 16 |
| | UUGGAUCCUCUGUAAGAGAUUACGGUUAUCUCCGUAUGAAA | |
| | CAGUUGUUUACCCUG | |
| R13-C37 | 5′GGGAGACCGGCCAGCAUCAUGCAUAUUGGCCAUUGCGUU | 17 |
| | GCUCGCACCUGGGGACCUGUGCGUUCCCGCCCACCGUGUCU | |
| | CCAAGGAUCCCUACA | |
| R21-49050 | 5′GGGAGACCGGCCAGCGUACACAACGCACCAGUCCAUGAGU | 18 |
| | UCGGUCUCGCCCGCAUAAUUCGCGCCAGCGCAGCACUCGAA | |
| | UUCAUUUUCCCUU | |
| R21-30360 | 5′GGGAGACCGGCCAGCGCUAUUGUGUCUCUCGAGCCUCUG | 19 |
| | UUAUCGCACGCCUUGAGUUGCACUUAGUCCGCAAAUGUCCA | |
| | GUGUUGUUCCCUUA | |

### Example 3. Determination of Functional Features of the Enriched Sequences Obtained with the Helicase SELEX Process

FIG. 8 presents the measurement of the Rho helicase activity in presence of enriched sequences from R13 library (C1, C2, C6, C37 and C39) and from R21 library (49050, 30360, 21625, 217173).

Duplexes containing the aRut or iRut sequence (Table 2) instead of the variable N₈₀ sequence of FIG. 5A are used in control experiments. In agreement with previous results, the aRut sequence is able to elicit a strong Rho helicase activity whereas the iRut sequence is not (5,12). The reactions with the control duplexes are not affected by the presence of 10 mM 5-HT (top line of FIG. 8).

The reaction time-course with the aRut duplex (dotted curve on all graphs of FIG. 8) is used to benchmark the efficiency of the various R13 and R21 sequences at triggering Rho helicase activity.

In the presence of 5-HT, only three of the tested sequences elicit an activity that is similar (R13-C37 and R21-49050) or even superior (R21-30360) to that measured with the aRut control (bottom lines of FIG. 8). In absence of 5-HT, these three sequences are poorly efficient at activating the Rho helicase, behaving similarly to the inactive iRut control.

Equilibrium binding measurements indicate that the presence of 5-HT significantly increases the affinity of Rho for the R13-C37, R21-30360 and R21-49050 duplexes (not shown), which is consistent with the model presented in FIG. 4 (bottom).

### Example 4. Selection of Natural Aptamers Substrates of Rho Enzyme with the Helicase-SELEX Process

A published procedure (2) was adapted to prepare DNA templates containing fragments of the Escherichia coli genome framed by fixed sequences. In the procedure, E. coli's DNA is amplified by PCR with a pair of partially randomized primers. The primers contain the FWD or REV sequence followed by a random sequence. The PCR products are purified by PAGE alongside a DNA size ladder; PCR products in the target size range are excised and eluted from the PAGE gel.

The resulting library of DNA templates is transcribed with T7 RNA polymerase to generate a library of ssRNA strands, each containing a natural sequence of ˜50 to ˜100 nt. The ssRNA strands are then hybridized with a biotinylated oligonucleotide and used in Helicase-SELEX process to seek natural aptamers of the Rho helicase.

To limit potential interference of the upstream ssRNA constant region upon pairing with the natural sequence, a complementary oligonucleotide is used to shield the constant region in the duplexes.

Preparation of this library in three steps 1, 2 and 3 is illustrated in FIG. 10A.

Measure of the dissociated fraction is presented in FIG. 10B, for R0 and R3 libraries.

The starting library of duplexes (R0) containing natural sequences is significantly more susceptible to Rho helicase activity than R0 library counterparts containing synthetic randomized sequences (compare the helicase activity elicited by the R0 library below with that of FIG. 2, for instance).

This is consistent with the fact that the E. coli genome encodes hundreds of Rut sites (18-20) that constitute a large reservoir of natural aptamers. It is possible to iteratively (and quickly) enrich the library in the best natural aptamers sequences, as shown by the increased reactivity displayed by the duplex library after only 3 rounds of Helicase-SELEX (R3)

### Example 5. Obtained Riboswitches are Sensitive to 5-HT in E. coli Cells

The control aRut and iRut sequences as well as the R21-30360 and R21-49050 riboswitch sequences were introduced in dual reporter plasmids (tsp-less series; see methods) between the GFP gene and its promoter (FIG. 7). A plasmid without any sequence insert was also used as control.

E. coli cells carrying the plasmids were grown in presence or absence of serotonin (5-HT). Cell cultures were analyzed by flow cytometry in order to determine the expression of the GFP reporter in each condition. The expression of the dsREDexpress2 reporter was used to normalize the GFP signal for variations in plasmid copy numbers.

As illustrated in FIG. 12, the normalized GFP signals of cells carrying the control plasmids (without insert or with the aRut or iRut insert) are not affected significantly by the presence of serotonin.

By contrast, the normalized GFP signals of cells carrying the R21-30360 and R21-49050 riboswitches significantly decrease in presence of serotonin. This is consistent with productive serotonin-dependent recruitment of the Rho factor on the mRNA by the riboswitches leading to a decrease of GFP expression upon Rho-dependent termination of transcription.

The R21-30360 construct is the most efficient, which agrees well with the superior kinetic behavior of the riboswitch (as is shown in FIG. 8), as well as with Rho-dependent termination being under kinetic control (21).

### REFERENCES

Patents
U.S. Pat. No. 5,475,096
U.S. Pat. No. 5,270,163
U.S. Pat. No. 5,707,796
U.S. Pat. No. 5,763,177
U.S. Pat. No. 6,001,577
U.S. Pat. No. 5,580,737
U.S. Pat. No. 5,567,588
U.S. Pat. No. 6,706,482

Scientific Publications
1. Hollands, K., Sevostiyanova, A. and Groisman, E. A. (2014) Unusually long-lived pause required for regulation of a Rho-dependent transcription terminator. Proc Natl Acad Sci USA, 111, E1999-2007.
2. Zimmermann, B., Bilusic, I., Lorenz, C. and Schroeder, R. (2010) Genomic SELEX: a discovery tool for genomic aptamers. Methods, 52, 125-132.
3. Boudvillain, M., Walmacq, C., Schwartz, A. and Jacquinot, F. (2010) Simple enzymatic assays for the in vitro motor activity of transcription termination factor Rho from Escherichia coli. Methods Mol Biol, 587, 137-154.
4. Neidhardt, F. C., Bloch, P. L. and Smith, D. F. (1974) Culture medium for enterobacteria. J Bacteriol, 119, 736-747.
5. Soares, E., Schwartz, A., Nollmann, M., Margeat, E. and Boudvillain, M. (2014) The RNA-mediated, asymmetric ring regulatory mechanism of the transcription termination Rho helicase decrypted by time-resolved Nucleotide Analog Interference Probing (trNAIP). Nucleic Acids Res, 42, 9270-9284.
6. Ruscito, A. and DeRosa, M. C. (2016) Small-Molecule Binding Aptamers: Selection Strategies, Characterization, and Applications. Front Chem, 4, 14.
7. Ozer, A., White, B. S., Lis, J. T. and Shalloway, D. (2013) Density-dependent cooperative non-specific binding in solid-phase SELEX affinity selection. Nucleic Acids Res, 41, 7167-7175.
8. Schwartz, A., Rabhi, M., Jacquinot, F., Margeat, E., Rahmouni, A. R. and Boudvillain, M. (2009) A stepwise 2′-hydroxyl activation mechanism for the bacterial transcription termination factor Rho helicase. Nat Struct Mol Biol, 16, 1309-1316.
9. Bendixsen, D. P., J, M. R., Townshend, B. and Hayden, E. J. (2020) Phased nucleotide inserts for sequencing low-diversity RNA samples from in vitro selection experiments. RNA, 26, 1060-1068.
10. Lutz, R. and Bujard, H. (1997) Independent and tight regulation of transcriptional units in Escherichia coli via the LacR/O, the TetR/O and AraC/I1-I2 regulatory elements. Nucleic Acids Res, 25, 1203-1210.
11. Guerin, M., Robichon, N., Geiselmann, J. and Rahmouni, A. R. (1998) A simple polypyrimidine repeat acts as an artificial Rho-dependent terminator in vivo and in vitro. Nucleic Acids Res., 26, 4895-4900.
12. Walmacq, C., Rahmouni, A. R. and Boudvillain, M. (2004) Influence of substrate composition on the helicase activity of transcription termination factor Rho: reduced processivity of Rho hexamers during unwinding of RNA-DNA hybrid regions. J Mol Biol, 342, 403-420.
13. Hollands, K., Proshkin, S., Sklyarova, S., Epshtein, V., Mironov, A., Nudler, E. and Groisman, E. A. (2012) Riboswitch control of Rho-dependent transcription termination. Proc Natl Acad Sci USA, 109, 5376-5381.
14. Ambriz-Avina, V., Contreras-Garduno, J. A. and Pedraza-Reyes, M. (2014) Applications of flow cytometry to characterize bacterial physiological responses. Biomed Res Int, 2014, 461941.
15. Schwartz, A., Walmacq, C., Rahmouni, A. R. and Boudvillain, M. (2007) Noncanonical interactions in the management of RNA structural blocks by the transcription termination rho helicase. Biochemistry, 46, 9366-9379.
16. D'Heygere, F., Schwartz, A., Coste, F., Castaing, B. and Boudvillain, M. (2015) Monitoring RNA Unwinding by the Transcription Termination Factor Rho from Mycobacterium tuberculosis. Methods Mol Biol, 1259, 293-311.
17. Rabhi, M., Espeli, O., Schwartz, A., Cayrol, B., Rahmouni, A. R., Arluison, V. and Boudvillain, M. (2011) The Sm-like RNA chaperone Hfq mediates transcription antitermination at Rho-dependent terminators. Embo J, 30, 2805-2816.
18. Peters, J. M., Mooney, R. A., Grass, J. A., Jessen, E. D., Tran, F. and Landick, R. (2012) Rho and NusG suppress pervasive antisense transcription in Escherichia coli. Genes Dev, 26, 2621-2633.
19. Magan, A., Amman, F., El-Isa, F., Hartl, N., Shamovsky, I., Nudler, E., Schroeder, R. and Sedlyarova, N. (2019) iRAPs curb antisense transcription in E. coli. Nucleic Acids Res, 47, 10894-10905.
20. Nadiras, C., Eveno, E., Schwartz, A., Figueroa-Bossi, N. and Boudvillain, M. (2018) A multivariate prediction model for Rho-dependent termination of transcription. Nucleic

Acids Res, 46, 8245-8260.
21. Jin, D. J., Burgess, R. R., Richardson, J. P. and Gross, C. A. (1992) Termination efficiency at rho-dependent terminators depends on kinetic coupling between RNA polymerase and rho. Proc Natl Acad Sci USA, 89, 1453-1457.
22. Jang K J, Lee N R, Yeo W S, Jeong Y J, Kim D E. Isolation of inhibitory RNA aptamers against severe acute respiratory syndrome (SARS) coronavirus NTPase/Helicase. Biochem Biophys Res Commun. 2008 Feb. 15; 366(3):738-44.
23. Skordalakes E, Berger JM. Structure of the Rho transcription terminator: mechanism of mRNA recognition and helicase loading. Cell. 2003 Jul. 11; 114(1):135-46.
24. Fiorini F, Bagchi D, Le Hir H, Croquette V. Human Upf1 is a highly processive RNA helicase and translocase with RNP remodelling activities. Nat Commun. 2015 Jul. 3; 6:7581.

1. Process for selecting aptamers substrates of one helicase enzyme, comprising the implementation of a helicase SELEX process comprising several cycles, wherein each cycle comprises the following steps:
a) Providing a library of nucleic acid duplex constructs comprising one nucleic acid strand containing a random sequence of 10 to 100 nucleotides framed by fixed sequences at each end, and one other nucleic acid strand;
b) Incubation of said library with said helicase in appropriate conditions for the dissociation of certain duplex constructs by the helicase, resulting in release of the aptamers substrates of the helicase;
c) Isolation and amplification of said aptamers substrates of the helicase;
d) Creation of a novel library of nucleic acid duplex constructs enriched in duplex constructs comprising aptamers substrates of the helicase.

2. Process according to claim 1, comprising at least five cycles, wherein after the step (c) of the last cycle, the aptamers substrates of the helicase are sequenced.

3. Process according to claim 1, wherein the nucleic acid duplex constructs are biotinylated and immobilized on streptavidin carrying beads.

4. Process for selecting switches stimulating the activity of one helicase enzyme in response to the presence of a specific inducer, comprising the implementation of a helicase SELEX process comprising several cycles, wherein each cycle comprises the following steps:
Providing a library of nucleic acid duplex constructs comprising one nucleic acid strand containing a random sequence of 10 to 100 nucleotides framed by fixed sequences at each end, and one other nucleic acid strand;
b1) Incubation of said library with said helicase and said specific inducer in appropriate conditions for the dissociation of certain duplex constructs by the helicase, resulting in release of the switches comprising the sequences that are substrates of the helicase in presence of said inducer; and/or
b2) incubation of said library with said helicase without any inducer for retention of switch-containing duplex constructs not dissociated by said helicase in absence of said inducer and elimination of duplex constructs dissociated by said helicase in absence of said inducer;
Isolation and amplification of said switches;
Creation of a novel library of nucleic acid duplex constructs enriched in duplex constructs comprising switches modulating the activity of the helicase in presence of a specific inducer and wherein at least one cycle comprises at least one step (b1).

5. Process according to claim 4, wherein the specific inducer is a natural inducer.

6. Process according to claim 4, wherein the concentration of the specific inducer is adjusted for improving the selection pressure of the selection process.

7. Process according to claim 4, wherein both steps (b1) and (b2) are carried out in any order.

8. Process according to claim 1, wherein the helicase enzyme is Rho or Upf1.

9. Process according to claim 1, wherein said aptamers substrates of the helicase are RNA aptamers and said switches are riboswitches, and the one nucleic acid strand containing a random sequence in the duplex constructs is a RNA strand.

10. Process according to claim 1, wherein the other nucleic acid strand of the duplex constructs is a DNA strand, a RNA strand, or a 2′-alkyl-RNA strand.

11. Process according to claim 1, wherein the steps of the helicase SELEX process are automatically implemented by a robot.

12. Isolated aptamer, substrate of a helicase, obtained by the process according to claim 1.

13. Isolated switch modulating the activity of a helicase in response to the presence of a specific inducer, obtained by the process according to claim 4.

14. Genetic construction comprising the switch of claim 13 and an expression cassette.

15. A method for detecting a compound of interest, using the reporter system of claim 14, wherein the switch is responsive to the presence of said compound of interest.

16. Process according to claim 4, wherein the helicase enzyme is Rho or Upf1.

17. Process according to claim 4, wherein switches are riboswitches, and the one nucleic acid strand containing a random sequence in the duplex constructs is a RNA strand.

18. Process according to claim 4, wherein the other nucleic acid strand of the duplex constructs is a DNA strand, a RNA strand, or a 2′-alkyl-RNA strand.

19. Process according to claim 4, wherein the steps of the helicase SELEX process are automatically implemented by a robot.

20. Process according to claim 5, wherein the specific inducer is serotonin or a synthetic inducer.

21. Genetic construction according to claim 14, which is a reporter system comprising a reporter gene in said expression cassette.


SEQUENCE LISTING (20 sequences)

SEQ ID NO: 1 - DNA, 99 bp, Artificial Sequence
  Randomized region framed by fixed sequences
  misc_feature (16)..(65): n is a, c, g, or t
gggagaccggccagcnnnnnnnnnnnnnnnnnnnnnnnnnnnnnnnnnnnnnnnnnnnnnnnnnncgatggtatcagatctggatcctcgagaagctgc

SEQ ID NO: 2 - DNA, 129 bp, Artificial Sequence
  Randomized region framed by fixed sequences
  misc_feature (16)..(95): n is a, c, g, or t
gggagaccggccagcnnnnnnnnnnnnnnnnnnnnnnnnnnnnnnnnnnnnnnnnnnnnnnnnnnnnnnnnnnnnnnnnnnnnnnnnnnnnnnnncgatggtatcagatctggatcctcgagaagctgc

SEQ ID NO: 3 - DNA, 38 bp, Artificial Sequence
  FWD
cgaaattaatacgactcactatagggagaccggccagc

SEQ ID NO: 4 - DNA, 57 bp, Artificial Sequence
  REV
cgatgaattcgagctcggtacccgcagcttctcgaggatccagatctgataccatcg

SEQ ID NO: 5 - DNA, 67 bp, Artificial Sequence
  SEL
ttttttttttcgatgaattcgagctcggtacccgcagcttctcgaggatccagatctgataccatcg

SEQ ID NO: 6 - DNA, 57 bp, Artificial Sequence
  TRAP
cgatggtatcagatctggatcctcgagaagctgcgggtaccgagctcgaattcatcg

SEQ ID NO: 7 - DNA, 33 bp, Artificial Sequence
  LESS-OLN
gggagaccggccagccgatggtatcagatctgg

SEQ ID NO: 8 - DNA, 56 bp, Artificial Sequence
  FORWA
cggctcgtataatgtgtggaattgtgagcggataacaatttgggagaccggccagc

SEQ ID NO: 9 - DNA, 53 bp, Artificial Sequence
  FORWB
gcggccgcactcgaggagctgttgacaattaatcatcggctcgtataatgtgt

SEQ ID NO: 10 - DNA, 44 bp, Artificial Sequence
  FACS-REV
actgtcttacacaccggtaagacagcccagatctgataccatcg

SEQ ID NO: 11 - DNA, 113 bp, Artificial Sequence
  C1-OLN
gggagaccggccagccccatgtatcgtcgagggcagttcttggatcctctgtaagagattacggttatctccgtatgaaacagttgtttaccctgcgatggtatcagatctgg

SEQ ID NO: 12 - RNA, 52 nt, Artificial Sequence
  aRut
acuucuccucugucuccuucuuccuucuccucugucuccuucuuccucgacc

SEQ ID NO: 13 - RNA, 52 nt, Artificial Sequence
  iRut
ggucgaggaagaaggagacagaggagaaggaagaaggagacagaggagaagu

SEQ ID NO: 14 - RNA, 64 nt, Artificial Sequence
  1-386
gggagaccggccagcguuuggcuggaauccguuucuuggcucauuucugggucaaauucucugu

SEQ ID NO: 15 - RNA, 65 nt, Artificial Sequence
  2-355
gggagaccggccagcuugggucggccaaucccgugucuugcaguauuuccacugcgaccuuccua

SEQ ID NO: 16 - RNA, 95 nt, Artificial Sequence
  R13-C1
gggagaccggccagccccauguaucgucgagggcaguucuuggauccucuguaagagauuacgguuaucuccguaugaaacaguuguuuacccug

SEQ ID NO: 17 - RNA, 95 nt, Artificial Sequence
  R13-C37
gggagaccggccagcaucaugcauauuggccauugcguugcucgcaccuggggaccugugcguucccgcccaccgugucuccaaggaucccuaca

SEQ ID NO: 18 - RNA, 94 nt, Artificial Sequence
  R21-49050
gggagaccggccagcguacacaacgcaccaguccaugaguucggucucgcccgcauaauucgcgccagcgcagcacucgaauucauuuucccuu

SEQ ID NO: 19 - RNA, 94 nt, Artificial Sequence
  R21-30360
gggagaccggccagcgcuauugugucucucgagccucuguuaucgcacgccuugaguugcacuuaguccgcaaauguccaguguuguucccuua

SEQ ID NO: 20 - DNA, 292 bp, Artificial Sequence
  pTac-sfGFP leader region with Rho-dependent transcription termination signal
ctcgaggagctgttgacaattaatcatcggctcgtataatgtgtggaattgtgagcggataacaatttgggagaccggccagcacttctcctctgtctccttcttccttctcctctgtctccttcttcctcgacccgatggtatcagatctgggctgtcttaccggtgtgtaagacagtgacacaataacgtccctgtttttatttaaacattgctcatcgggcaaggctttgccgtgcctgaagaattttctgcgcctgacttcggcgcatctagaaggagatataccatg